(19) United States
(12) Patent Application Publication
(10) Pub. No.: US 2022/0296503 A1
(43) Pub. Date: Sep. 22, 2022

(54) DELAYING PEAK EFFECT AND/OR EXTENDING DURATION OF RESPONSE

(71) Applicant: Eirion Therapeutics, Inc., Woburn, MA (US)

(72) Inventor: Jonathan Edelson, Nashua, NH (US)

(21) Appl. No.: 17/610,057

(22) PCT Filed: May 12, 2020
(86) PCT No.: PCT/US20/32458

§ 371 Date: Nov. 9, 2021

Related U.S. Application Data
(60) Provisional application No. 62/847,901, filed on May 14, 2019.

(51) Int. Cl. A61K 9/00; A61K 47/42; A61K 38/48; A61K 38/39; A61K 31/80; A61K 31/573; A61K 31/192; A61K 31/167; C07K 16/24; C07K 16/28; A61M 37/00

(52) U.S. Cl. CPC A61K 9/0021; A61K 47/42; A61K 38/4893; A61K 38/39; A61K 31/80; A61K 31/573; A61K 31/192; A61K 31/167; C07K 16/241; C07K 16/2806; C07K 16/2812; C07K 16/244; A61M 37/0015; A61M 2037/0023; A61M 2037/0061

(57) ABSTRACT

The present disclosure describes certain technologies for administering large agent(s) (e.g., biologically active large agents such as biologic therapeutics including, for instance, botulinum toxin) to subjects, including technologies that achieve unexpected results such as, for example, delayed peak effect and/or extended duration of response.

### CROSS-REFERENCE TO RELATED APPLICATIONS

This application claims priority to U.S. Provisional Patent Application No. 62/847,901, filed May 14, 2019, the entire contents of which are hereby incorporated by reference in their entirety.

### BACKGROUND

Significant resources are invested in development of technologies for administration of large agents (e.g., biologically active large agents).

### SUMMARY

The present disclosure describes certain technologies for administering large agent(s) (e.g., biologically active large agents such as biologic therapeutics including, for instance, botulinum toxin) to subjects, and furthermore describes unexpected results achieved by such technologies including, for example, delayed peak effect and/or extended duration of response. In some embodiments, such unexpected results are achieved relative to administration of the same large agent (e.g., botulinum toxin) according to a reference regimen (e.g., via a formulation, route, and/or dosing regimen understood in the art). Those skilled in the art will be aware of relevant (e.g., appropriately comparable) reference regimens, including those for approved products as described herein. Moreover, those skilled in the art will appreciate that, in some embodiments, an appropriate reference regimen is one that achieves a peak effect that is comparable in magnitude to that of a provided regimen, and furthermore will be familiar with comparing regimens (and/or effects of regimens) that may involve administration of a relevant large agent (e.g., botulinum toxin) via different formulations, different dose amounts, different number of doses, different spacing between doses, different routes of administration, etc. To give but one example, those skilled in the art are aware that individual doses of botulinum toxin administered parenterally are typically much lower (i.e., contain many fewer units of botulinum toxin) than are individual doses of botulinum toxin administered topically [because topical administration of any active ingredient typically only delivers a small percentage of the active into the skin whereas parental administration delivers one hundred percent into the skin]; those skilled in the art are nonetheless able to assess comparable parenteral vs topical regimens, and to compare parameters (e.g., one or more outcome parameters) relevant to the present disclosure, such as timing of peak effect and duration of effect, etc., for example as may be as assessed based on the agents' biologic effects (e.g., reduction of facial wrinkles at maximal contraction as measured by a physician using wrinkle assessment scales, etc).

Among other things, the present disclosure identifies the source of a problem with certain conventional technologies for administration of large agents, and particularly for administration of botulinum toxin, including for example that many such technologies are designed to achieve rapid onset and/or rapid peak effect. The present disclosure teaches, among other things, that in certain circumstances (including, e.g., for many subjects receiving treatment for wrinkles), a delayed onset and/or delayed peak effect may be desirable and/or beneficial. The present disclosure furthermore documents that provided technologies can achieve a surprising and unexpected a delayed onset and/or delayed peak effect.

Furthermore, the present disclosure demonstrates that provided technologies can surprisingly achieve a surprising and unexpected extended duration of effect. Among other things, the present disclosure appreciates that such extended duration of effect may have a variety of beneficial effects including, for example, permitting less frequent administration of doses which may in turn provide greater convenience (e.g, fewer trips to a doctor's office or clinic) and/or cost savings.

Technologies provided herein particularly include transdermal delivery technologies. Those skilled in the art are well aware of challenges associated with achieving effective transdermal delivery, particularly for large agents. It has generally been understood that, as molecular size increases, transdermal penetration decreases, to the point where it is de minimis and even non-existent. The present disclosure provides particular technologies in which topical application of a large agent formulation (e.g., a nanoemulsion formulation), in combination with microneedle skin conditioning (e.g., pre-conditioning), achieve surprising and unexpected results, which may, in some embodiments, include one or both of delayed peak effect and/or extended duration of response.

### BRIEF DESCRIPTION OF THE DRAWING

FIG. 1: FIG. 1 presents a bar graph comparing responder rates observed with provided technologies as compared with an approved injectable botulinum toxin therapy, and illustrates, for example, an extended duration of effect achieved by provided technologies as described herein.

FIG. 2: FIG. 2 presents a line graph comparing timing of peak effect for two different approved injectable botulinum toxin therapies with provided topical+MSC therapies and documents, for example, a delayed peak effect achieved by provided technologies as described herein.

### DEFINITIONS

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

Abrasion: The term “abrasion,” as used herein refers to any means of altering, disrupting, removing, or destroying the top layer of the skin. In some embodiments, abrasion refers to a mechanical means of altering, disrupting, removing, or destroying the top layer of the skin. In some embodiments, abrasion refers to a chemical means of altering, disrupting, removing, or destroying the top layer of skin. To give but a few examples, agents such as exfoliants, fine particles (e.g. magnesium or aluminum particles), acids (e.g alpha-hydroxy acids or beta-hydroxy acids), alcohols, may cause abrasion. In general, permeation enhancers such as those described, for example, by Donovan (e.g. US Publications 2004/009180 and 2005/175636, and PCT Publication WO 04/06954), and Graham (e.g. U.S. Pat. No. 6,939,852 and US Publication 2006/093624), etc., are expected to cause abrasion. Of course, those of ordinary skill in the art will appreciate that a particular agent may cause abrasion when present at one concentration, or in association with one or more other agents, but may not cause abrasion under different circumstances. Thus, whether or not a particular material is an “abrasive agent” depends on context. Abrasion can readily be assessed by those of ordinary skill in the art, for example by observation of redness or irritation of the skin and/or histologic examination of skin showing alteration, disruption, removal, or erosion of the stratum corneum.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be parenteral; in some embodiments, administration may be topical. In some embodiments, administration may involve dosing that is intermittent (e.g., a plurality of doses separated in time) and/or periodic (e.g., individual doses separated by a common period of time) dosing. In some embodiments, administration may involve continuous dosing for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments; the term may refer to a compound or entity that lacks or is substantially free of any polymeric moiety. In some embodiments, the term may refer to a molecular complex. In many embodiments as described herein, an agent may be a large agent (e.g., a biologically active large agent such as a biologic therapeutic including, for example, botulinum toxin).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kDa tetrameric agents comprised of two identical heavy chain polypeptides (about 50 kDa each) and two identical light chain polypeptides (about 25 kDa each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™”), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification e.g., attachment of a glycan) that it would have if produced naturally (e.g., in a mammalian organism). In some embodiments, an antibody may contain a covalent modification e.g, attachment of a glycan, a payload [e.g., a detectable moiety, a therapeutic moiety, a catalytic moiety, etc], or other pendant group [e.g., poly-ethylene glycol, etc.]

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplar)/antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is or comprises an antibody-drug conjugate.

Antibody component: as used herein, refers to a polypeptide element (that may be a complete polypeptide, or a portion of a larger polypeptide, such as for example a fusion polypeptide as described herein) that specifically binds to an epitope or antigen and includes one or more immunoglobulin structural features. In general, an antibody component is any polypeptide whose amino acid sequence includes elements characteristic of an antibody-binding region (e.g., an antibody light chain or variable region or one or more complementarity determining regions (“CDRs”) thereof, or an antibody heavy chain or variable region or one more CDRs thereof, optionally in presence of one or more framework regions). In some embodiments, an antibody component is or comprises a full-length antibody. In some embodiments, an antibody component is less than full-length but includes at least one binding site (comprising at least one, and preferably at least two sequences with structure of known antibody “variable regions”). In some embodiments, the term “antibody component” encompasses any protein having a binding domain, which is homologous or largely homologous to an immunoglobulin-binding domain. In particular embodiments, an included “antibody component” encompasses polypeptides having a binding domain that shows at least 99% identity with an immunoglobulin binding domain. In some embodiments, an included “antibody component” is any polypeptide having a binding domain that shows at least 70%, 75%, 80%, 85%, 90%, 95% or 98% identity with an immunoglobulin binding domain, for example a reference immunoglobulin binding domain. An included “antibody component” may have an amino acid sequence identical to that of an antibody (or a portion thereof, e.g., an antigen-binding portion thereof) that is found in a natural source. An antibody component may be monospecific, bi-specific, or multi-specific. An antibody component may include structural elements characteristic of any immunoglobulin class, including any of the human classes: IgG, IgM, IgA. IgD, and IgE. It has been shown that the antigen-binding function of an antibody can be performed by fragments of a full-length antibody. Such antibody embodiments may also be bispecific, dual specific, or multi-specific formats specifically binding to two or more different antigens. Examples of binding fragments encompassed within the term “antigen-binding portion” of an antibody include (i) a Fab fragment, a monovalent fragment consisting of the V_{H}, V_{L}, C_{H}1 and C_{L} domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{H} and V_{L} domains of a single arm of an antibody, (v) a dAb fragment (Ward et al., (1989) Nature 341:544-546), which comprises a single variable domain; and (vi) an isolated complementarily determining region (CDR). Furthermore, although the two domains of the Fv fragment, V_{H} and V_{L}, are coded for by separate genes, they can be joined, using recombinant methods, by a synthetic linker that enables them to be made as a single protein chain in which the V_{H} and V_{L} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). In some embodiments, an “antibody component”, as described herein, is or comprises such a single chain antibody. In some embodiments, an “antibody component” is or comprises a diabody. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al., (1993) Proc. Natl. Acad. Sci. USA 90:6444-6448; Poljak, R. J., (1994) Structure 2(12):1121-1123). Such antibody binding portions are known in the art (Kontermann and Dubel eds., Antibody Engineering (2001) Springer-Verlag. New York. 790 pp. (ISBN 3-540-41354-5). In some embodiments, an antibody component is or comprises a single chain “linear antibody” comprising a pair of tandem Fv segments (V_{H}-C_{H}1-V_{H}-C_{H}1) which, together with complementary light chain polypeptides, form a pair of antigen binding regions (Zapata et al., (1995) Protein Eng. 8(10): 1057-1062; and U.S. Pat. No. 5,641,870). In some embodiments, an antibody component may have structural elements characteristic of chimeric or humanized antibodies. In general, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some embodiments, an antibody component may have structural elements characteristic of a human antibody.

Antibody fragment: As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region. An antigen binding fragment of an antibody may be produced by any means. For example, an antigen binding fragment of an antibody may be enzymatically or chemically produced by fragmentation of an intact antibody and/or it may be recombinantly produced from a gene encoding the partial antibody sequence. Alternatively or additionally, antigen binding fragment of an antibody may be wholly or partially synthetically produced. An antigen binding fragment of an antibody may optionally comprise a single chain antibody fragment. Alternatively or additionally, an antigen binding fragment of an antibody may comprise multiple chains which are linked together, for example, by disulfide linkages. An antigen binding fragment of an antibody may optionally comprise a multimolecular complex. A functional antibody fragment typically comprises at least about 50 amino acids and more typically comprises at least about 200 amino acids.

Approximately: As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (for example when the one or more values of interest define a sufficiently narrow range that application of such a percentage variance would obviate the stated range).

Associated with: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biocompatible: The term “biocompatible”, as used herein, refers to materials that do not cause significant harm to living tissue when placed in contact with such tissue, e.g., in vivo. In some embodiments, materials are “biocompatible” if they are not toxic to cells. In some embodiments, materials are “biocompatible” if their addition to cells in vitro results in less than or equal to 20% cell death, and/or their administration vivo does not induce significant inflammation or other such adverse effects.

Biodegradable: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In some embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Biologically active agent: As used herein, the term “biologically active agent” refers to an agent that has a particular biological effect when administered to a subject, e.g., a human. In some embodiments, a biologically active agent may be a therapeutically active agent, a cosmetically active agent, and/or a diagnostically active agent. In some embodiments, a biologically active agent may be or comprise an entity or moiety that would be classified as an “Active Pharmaceutical Ingredient” by the United States Food and Drug Administration. In some embodiments, a biologically active agent is a large agent. In some embodiments, a biologically active agent may be or comprise an agent whose presence correlates with a desired pharmacologic and/or therapeutic, cosmetic, and/or diagnostic effect. In some embodiments, a biologically active agent is characterized in that its biological effect is dose-dependent (e.g., increases with increasing dose, optionally in a linear manner over at least a first range of concentrations).

Botulinum macroemulsion composition: The term “botulinum macroemulsion composition.” as used herein, refers to a macroemulsion composition in which at least one macroemulsion includes a botulinum toxin. The botulinum toxin may be present within the macroemulsion, on the macroemulsion surface and/or within a micellar membrane defining the macroemulsion.

Botulinum nanoemulsion composition: The term “botulinum nanoemulsion composition,” as used herein, refers to a nanoemulsion composition in which at least one nanoemulsion includes a botulinum toxin. The botulinum toxin may be present within the nanoemulsion, on the nanoemulsion surface and/or within a micellar membrane defining the nanoemulsion.

Botulinum toxin: The term “botulinum toxin,” as used herein, refers to an neurotoxin produced by Clostridium botulinum. Except as otherwise indicated, the term encompasses fragments or portions (e.g., the light chain and/or the heavy chain) of such neurotoxin that retain appropriate activity (e.g., muscle relaxant activity). The phrase “botulinum toxin,” as used herein, may refer to a botulinum toxin or serotype A, B, C, D, E, F, or G. As will be understood by, those skilled in the art, in some embodiments, the term botulinum toxin, as used herein, can encompass a botulinum toxin complex (i.e., for example, the 300, 600, and 900 kDa complexes) or a purified (i.e., for example, isolated) botulinum toxin (i.e., for example, about 150 kDa). “Purified botulinum toxin” is understood to be a botulinum toxin that is isolated, or substantially isolated, from other proteins, e.g., proteins with which a botulinum toxin may be associated in nature, including those that participate in a botulinum toxin complex. A purified toxin may be greater than 95% pure, and in some embodiments is greater than 99% pure. Those of ordinary skill in the art will appreciate that the present invention is not limited to any particular source of botulinum toxin. For example, botulinum toxin for use in accordance with the present invention may be isolated from Clostridium botulinum, may be chemically synthesized, may be produced recombinantly (i in a host cell or organism other than Clostridium botulinum), etc. The botulinum toxin may be genetically engineered or chemically modified to act longer or shorter in duration than botulinum toxin serotype A.

Carrier: as used herein, refers to a diluent, adjuvant, excipient, or vehicle with which a composition is administered (e.g., that is a component of a formulation of the composition). In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents, a therapeutic agent and a therapeutic modality, etc.). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agents and/or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition”, as used herein, may be used to refer to a discrete physical entity that comprises one or more specified components. In general, unless otherwise specified, a composition may be of any form—e.g., gas, gel, liquid, solid, etc.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length, in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Emulsion: The term “emulsion” is used herein consistent with the understanding in the art of “a system . . . consisting of a liquid dispersed with or without an emulsifier in an immiscible liquid usually in droplets of larger than colloidal size”. See, for example, definition in Medline Plus Online Medical Dictionary, Merriam Webster (2005).

Excipient: as used herein, refers to a non-therapeutic agent that may be included in a pharmaceutical composition, for example to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Human: In some embodiments, a human is an embryo, a fetus, an infant, a child, a teenager, an adult, or a senior citizen.

Hydrophilic: As used herein, the term “hydrophilic” and/or “polar” refers to a tendency to mix with, or dissolve easily in, water.

Hydrophobic: As used herein, the term “hydrophobic” and/or “non-polar”, refers to a tendency to repel, not combine with, or an inability to dissolve easily in, water.

Improve, increase or reduce: As used herein or grammatical equivalents thereof, the terms “improve”, “increase” or “reduce” indicate values that are relative to a baseline measurement, such as a measurement in the same individual prior to initiation of a treatment described herein, or a measurement in a control individual (or multiple control individuals) in the absence of the treatment described herein. In some embodiments, a “control individual” is an individual afflicted with the same form of disease or injury as an individual being treated.

Large molecule: The term “large molecule” is generally used herein to describe a molecule that is greater than about 100 kilodaltons (KDa) in size. In some embodiments, a large molecule is greater than about 110 KDa, 120 KDa, 130 KDa, 140 KDa, 150 KDa, 160 KDa, 170 KDa, 180 KDa, 190 KDa, 200 KDa, 250 KDa, 300 KDa, 400 KDa, or 500 KDa. In some embodiments, a large molecule is a polymer or comprises a polymeric moiety or entity. In some embodiments, a large molecule is or comprises a polypeptide. In some embodiments, a large molecule is or comprises a nucleic acid.

Large agent: The term “large agent” as used herein generally refers to an agent having a molecular weight that is greater than about 100 kilodaltons (KDa) in size. In some embodiments, a large molecule is greater than about 110 KDa, 120 KDa, 130 KDa, 140 KDa, 150 KDa, 160 KDa, 170 KDa, 180 KDa, 190 KDa, 200 KDa, 250 KDa, 300 KDa, 400 KDa, or 500 KDa. In some embodiments, a large agent is a biologically active agent. In some embodiments, a large agent is or comprises one or more large molecules. In some embodiments, a large agent is or comprises one or more molecular complexes. In some embodiments, a large agent is or comprises a polypeptide. In some embodiments, a large agent is or comprises a complex of polypeptides. In some embodiments, a large agent is or comprises a bacterial toxin (e.g., a botulinum toxin). In some embodiments, a large agent is or comprises an antibody agent.

Macroemulsion: The term “macroemulsion,” as used herein, refers to an emulsion in which at least some droplets have diameters in the several hundred nanometers to micrometers size range. As will be understood by those of ordinary skill in the art, a macroemulsion is characterized by droplets greater than 300 nm in diameter. In some embodiments, a macroemulsion composition utilized in accordance with the present disclosure includes one or more large agents or one or more biologically active agents. In some embodiments, a large agent included in a macroemulsion composition may be a biologically active agent. It will be appreciated by those of ordinary skill in the art that a macroemulsion composition for use in accordance with the present disclosure may be prepared according to any available means including, for example, chemical or mechanical means. In some embodiments, droplets in a microemulsion have a size within a range of about 301 nm and about 1000 μm in some embodiments, a macroemulsion has droplets in a size distribution of between about 301 nm and about 1000 μm. In some embodiments, droplets in a macroemulsion have a size within a range of about 500 nm and about 5000 μm. In some embodiments, a macroemulsion has droplets in a size distribution of between about 500 nm and about 5000 μm.

Microneedle: The term “microneedle” as used herein generally refers to an elongated structure that is of suitable length, diameter, and shape to penetrate skin. In some embodiments, a microneedle is arranged and constructed (by itself or within a device) to minimize contact with nerves when inserted into skin, while still creating efficient pathways for drug delivery. In some embodiments, a microneedle has a diameter which is consistent along the microneedle's length. In some embodiments, a microneedle has a diameter that changes along the microneedle's length. In some embodiments, a microneedle has a diameter that tapers along the microneedle's length. In some embodiments, a microneedle's diameter is narrowest at the tip that penetrates skin. In some embodiments, a microneedle may be solid. In some embodiments, a microneedle may be hollow. In some embodiments a microneedle may be tubular. In some embodiments, a microneedle may be sealed on one end. In some embodiments, a plurality of microneedles is utilized. In some embodiments, a plurality of microneedles is utilized in an array format. In some embodiments, a microneedle may have a length within a range of about 1 μm to about 4,000 μm. In some embodiments, a microneedle may have a length of between about 1 μm to about 2,000 μm. In some embodiments, a microneedle may have a length of between about 50 μm to about 400 μm. In some embodiments, a microneedle may have a length of between about 800 μm to about 1500 μm.

Microneedle array impression: The term “microneedle array impression”, as used herein, refers to a microneedle impression achieved by impressing a microneedle and/or microneedle array onto skin and then removing it from the skin. In some embodiments, a microneedle array may be stamped onto skin (e.g., with a microneedle array stamp). In some embodiments, the microneedle array may be rolled onto skin (e.g., with a microneedle array roller).

Microneedle density: The term “microneedle density”, as used herein, refers to a number of microneedles per measure of area (e.g., square centimeters). In some embodiments, microneedle density is assessed as the number of microneedles per area of a microneedle array; in some embodiments, microneedle density is assessed as the number of microneedle punctures per area of microneedled site; in some embodiments, microneedle density is assessed as the number of microneedles per area that simultaneously achieve maximal or near maximal skin penetration possible for the microneedles in the array. Regardless, those of ordinary skill in the art will appreciate that microneedle density can be expressed whether the relevant area area is flat (e.g., microneedle array stamp), curved (e.g., microneedle array roller), or irregular. Those skilled in the art will appreciate that assessment of microneedle density as microneedle punctures per area of microneedled site may be particularly useful, for example, if an array has needles of different lengths and/or a microneedled site has topological variety such that not every needle may in fact puncture the skin when the array is applied to the site.

Microneedle puncture size: The term “microneedle puncture size” or “microneedle hole puncture size”, as used herein, refers to a calculated puncture area created by each microneedle of a microneedle array achieved after impressing the microneedle and/or microneedle array onto the skin and then removing it from the skin. In most embodiments the microneedle puncture size is calculated as the area of the base of the microneedle.

Nanoemulsion: The term “nanoemulsion,” as used herein, refers to an emulsion in which at least some droplets have diameters in the nanometer size range. As will be understood by those of ordinary skill in the art, a nanoemulsion is characterized by droplets 300 nm or smaller in diameter. In some embodiments, a nanoemulsion composition utilized in accordance with the present disclosure includes one or more large agents or one or more biologically, active agents. In some embodiments, a large agent included in a nanoemulsion composition may be a biologically active agent. It will be appreciated by those of ordinary skill in the art that a nanoemulsion composition for use in accordance with the present disclosure may be prepared according to any available means including, for example, chemical or mechanical means. In some embodiments, droplets in a nanoemulsion have a size within a range of about 1 nm and about 300 nm. In some embodiments, a nanoemulsion has droplets in a size distribution of between about 1 nm and about 300 mm.

Nanoparticle: As used herein, the term “nanoparticle” refers to a solid particle having a diameter of less than 300 nm, as defined by the National Science Foundation. In some embodiments, a nanoparticle has a diameter of less than 100 nm as defined by the National Institutes of Health.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Penetration enhancing agent: As used herein, the term “penetration enhancing agent” refers to an agent whose presence or level correlates with increased penetration of an agent of interest across skin, as compared with that observed in its absence. In some embodiments, a penetration enhancing agent is characterized in that it degrades and/or disrupts skin structure. In some embodiments, a penetration enhancing agent is or comprises a chemical agent (e.g., a chemical or enzyme, for example) For example, chemical agents that that may damage, disrupt, and/or degrade one or more stratum corneum components) may include, for example, alcohols, such as short chain alcohols, long chain alcohols, or polyalcohols; amines and amides, such as urea, amino acids or their esters, amides, AZONE®, derivatives of AZONE®, pyrrolidones, or derivatives of pyrrolidones; terpenes and derivatives of terpenes; fatty acids and their esters; macrocyclic compounds; tensides; or sulfoxides (e.g., dimethylsulfoxide (DMSO), decyhnethylsulfoxide, etc.); surfactants, such as anionic, cationic, and nonionic surfactants; polyols; essential oils; and/or hyaluronidase. In some embodiments, a penetration enhancing agent may be an irritant in that an inflammatory and/or allergic reaction occurs when the agent is applied to skin. In some embodiments, a penetration enhancing agent is not an irritant. In some embodiments, a penetration enhancing agent may be or comprise a chemical agent that does not damage, disrupt, or degrade skin structure but whose presence or level nonetheless correlates with increased penetration of an agent of interest across skin, as compared with that observed in its absence. In some embodiments, co-peptides, carrier molecules, and carrier peptides may be penetration enhancing agents which do not damage, disrupt, and/or degrade skin structure(s), In some embodiments, co-peptides, carrier molecules, and carrier peptides may be penetration enhancing agents which do not irritate the skin. The term “penetration enhancing agent” does not encompass mechanical devices (e.g., needles, scalpels, etc), or equivalents thereof (e.g., other damaging treatments). Also, those skilled in the art will appreciate that a structure such as a nanoparticle or an emulsion is not a chemical agent and therefore not a chemical penetration enhancing agent even if its presence correlates with enhanced skin penetration of an agent of interest that may be associated with the structure.

Pharmaceutical composition: As used herein, the term “pharmaceutical composition” refers to a composition in which an active agent is formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments; a pharmaceutical composition may be specially formulated for administration in solid or liquid form, including those adapted for topical administration, for example, a sterile solution or suspension, or sustained-release formulation, as a gel, cream, ointment; or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein; the term “pharmaceutically acceptable” applied to a carder, diluent, or excipient used to formulate a composition as disclosed herein means that the carrier, diluent, or excipient must be compatible with other ingredients of the composition and not deleterious to a recipient thereof.

Pharmaceutically acceptable carrier: As used herein, the term “pharmaceutically acceptable carrier” means a pharmaceutically-acceptable material, composition or vehicle, such as a liquid or solid tiller, diluent, excipient, or solvent encapsulating material, involved in carrying or transporting a subject compound from one organ, or portion of the body, to another organ, or portion of the body. Each carrier must be “acceptable” in the sense of being compatible with other ingredients of the formulation and not injurious to a subject or patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, medium chain triglycerides, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Premix: The term “premix” as used herein, refers to a combination of components that is subsequently used to generate an emulsion composition (e.g., a nanoemulsion composition). For example, in some embodiments, a premix is a collection of ingredients that, when subjected to high shear force, generates a nanoemulsions useful in accordance with the present disclosure. In some embodiments, a premix is a collection of ingredients that, when subjected to high shear force, generates a uniform nanoemulsions. A premix often contains a liquid dispersion medium and other components sufficient to generate nanoemulsion within the dispersion medium. According to some embodiments of the present disclosure, one or more large agents may be included in a premix. According to some embodiments of the present disclosure, one or more biologically agents may be included in a premix. According to the present invention, botulinum toxin may be included in a premix. According to the present invention, one or more antibodies may be included in a premix. In some embodiments, a premix may contain one or more surfactants, penetrating enhancers, and/or other agents. In some embodiments, a premix comprises a solution. In some embodiments in which a premix comprises botulinum toxin, an antibody, another biologically active agent and/or penetration enhancing agent, the botulinum toxin, the antibody, another biologically active agent and/or penetration enhancing agent, is in solution before high shear force is applied to the premix.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids, in some embodiments, proteins are antibodies, antibody fragments, biologically active portions thereof, and/or characteristic portions thereof.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, regimen, sequence or value of interest is compared with a reference or control agent, animal, individual, population, sample, regimen, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Self-administration: The term “self-administration,” as used herein, refers to the situation where a subject has the ability to administer a composition to him or herself without requiring medical supervision. In some embodiments of the invention, self-administration may be performed outside of a clinical setting. To give but one example, in some embodiments of the invention, a facial cosmetic cream may be administered by a subject in one's own home.

Small Molecule: In general, a “small molecule” is understood in the art to be an organic molecule that is less than about 5 kilodaltons (Kd) in size. In some embodiments, a small molecule is less than about 3 Kd, 2 Kd, or 1 Kd. In some embodiments, a small molecule is less than about 800 daltons (D), 600 D, 500 D, 400 D, 300 D, 200 D, or 100 D. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Subject: As used herein “subject” means an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, an appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, an agent is not considered to be a “therapeutic agent” if it merely enhances delivery of a different agent that in fact achieves the desired effect.

Therapeutically effective amount: As used herein, is meant an amount that produces a desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of a disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by a disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Therapeutic regimen: A “therapeutic regimen”, as that term is used herein, refers to a dosing regimen whose administration across a relevant population may be correlated with a desired or beneficial therapeutic outcome.

Treatment: As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Uniform: The term “uniform,” when used herein in reference to a nanoemulsion composition, refers to a nanoemulsion composition in which individual droplets have a specified range of droplet diameter sizes. For example, in some embodiments, a uniform nanoemulsion composition is one in which the difference between the minimum diameter and maximum diameter does not exceed approximately 300, 250, 200, 150, 100, 90, 80, 70, 60, 50, or fewer nm. In some embodiments, droplets (e.g., large agent-containing droplets) within inventive uniform large agent nanoemulsion compositions have diameters that are smaller than about 300, 250, 200, 150, 130, 120, 115, 110, 100, 90, 80 nm, or less. In some embodiments, droplets (e.g., large agent-containing droplets) within inventive uniform large agent nanoemulsion compositions have diameters within a range of about 10 and about 300 nanometers. In some embodiments, droplets within inventive uniform large agent nanoemulsion compositions have diameters within a range of about 10-300, 10-200, 10-150, 10-130, 10-120, 10-115, 10-110, 10-100, or 10-90 nm. In some embodiments, droplets (e.g., large agent-containing droplets) within inventive large agent nanoemulsion compositions have an average droplet size that is under about 300, 250, 200, 150, 130, 120, or 115, 110, 100, or 90 nm. In some embodiments, the average droplet size is within a range of about 10-300, 50-250, 60-200, 65-150, 70-130 nm. In some embodiments, the average droplet size is about 80-110 nm. In some embodiments, the average droplet size is about 90-100 nm. In some embodiments, a majority of droplets (e.g., large agent-containing droplets) within inventive uniform nanoemulsion compositions have diameters below a specified size or within a specified range. In some embodiments, a majority is more than 50%, 60%, 70%, 75%, 80%, 85%, 90%, 95%, 96%, 97%, 98%, 99%, 99.5%, 99.6%, 99.7%, 99.8%, 99.9% or more of the droplets in the composition. In some embodiments of the invention, a uniform nanoemulsion composition is achieved by microfluidization of a sample.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%, optionally other than conservative amino acid substitutions. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in a variant are substituted as compared with the parent. In some embodiments, a variant has 10, 9, 8, 7, 6, 5, 4, 3, 2, or 1 substituted residue(s) as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, a parent or reference polypeptide is one found in nature.

### DETAILED DESCRIPTION OF CERTAIN EMBODIMENTS

### Transdermal Drug Delivery

In some embodiments, the present disclosure provides technologies for improving delivery and/or bioavailability of large agents (e.g., botulinum toxin, antibodies) transdermally. In some embodiments, the present disclosure teaches that particularly advantageous results are achieved when microneedling technologies are combined with emulsion compositions. In some embodiments, microneedling technologies are combined with lotion, cream, or liquid compositions; in some embodiments, such compositions in turn may be or comprise emulsion compositions (e.g., macroemulsion compositions, microemulsion compositions, and/or nanoemulsion compositions). Alternatively, in some embodiments, provided technologies combine microneedling technologies with transdermal delivery that does not utilize a nanoemulsion, does not utilize a microemulsion, does not utilize a macroemulsion, or even does not utilize any emulsion. In some embodiments, provided technologies do not utilize penetration enhancing agents. In some embodiments, provided technologies do not utilize chemical penetration enhancing agents which damage, disrupt, and/or degrade the skin. In some embodiments, provided technologies do not utilize chemical penetration enhancing agents.

Human skin comprises the dermis and the epidermis. The epidermis has several layers of tissue, namely, stratum corneum, stratum lucidum, stratum granulosum, stratum spinosum, and stratum basale (identified in order from the outer surface of the skin inward).

The stratum corneum presents the most significant hurdle in transdermal delivery generally, and presumably of large agents in particular. The stratum corneum is typically about 10-15 μm thick, and it consists of flattened, keratised cells (corneocytes) arranged in several layers. The intercellular space between the corneocytes is filled with lipidic structures, and may play an important role in the permeation of substances through skin (Bauerova et al., 2001. European Journal of Drug Metabolism and Pharmacokinetics, 26:85).

The rest of the epidermis below the stratum corneum is approximately 150 μm thick. The dermis is about 1-2 mm thick and is located below the epidermis. The dermis is innervated by various capillaries as well as neuronal processes.

Transdermal administration generally has been the subject of research in attempts to provide an alternative route of administration without undesirable consequences associated with injections and oral delivery. For example, needles often cause localized pain, and potentially expose patients receiving injections to blood borne diseases. Oral administration often suffers from poor bioavailability of medications due to the extremely acidic environment of the patient's stomach.

Efforts have been made to develop transdermal administration techniques for certain pharmaceuticals in an attempt to overcome these shortcomings by providing noninvasive administration. It is generally desirable with transdermal administration to minimize damage to a patient's skin. Thus, transdermal administration may reduce or eliminate pain associated with injections, reduce the likelihood of blood contamination, and improve bioavailability of drugs once they are incorporated systemically.

Traditionally, attempts at transdermal administration have been focused on disruption and/or degradation of the stratum corneum. Some attempts have included using chemical penetration enhancing agents. Penetration enhancing agents may function to degrade and/or disrupt skin structure. In some embodiments, a penetration enhancing agent is or comprises a chemical agent (e.g., a chemical or enzyme, for example that may disrupt and/or degrade one or more stratum corneum components). In some embodiments, a penetration enhancing agent may be an irritant in that an inflammatory and/or allergic reaction occurs when the agent is applied to skin.

“However, the major limitation for penetration enhancers is that their efficacy is often closely correlated with the occurrence of skin irritation.” Alkilani, A. Z., et al., “Transdermal drug delivery: Innovative pharmaceutical developments based on disruption of the barrier properties of the stratum corneum.” Pharmaceutics. 7:438-470 (2015), Penetration enhancing agents tend to have poor efficacy and safety profiles. “They do not achieve the desired skin disruption and their ability to increase transport across the skin is low and variable.” Id.

Some attempts have included using mechanical apparatus to bypass or ablate portions of the stratum corneum. In addition, attempts have included use of ultrasound or iontophoresis to facilitate the penetration of pharmaceuticals through the skin. In most cases, the goal has been to enable a pharmaceutical agent, typically a small molecule, so that the agent may pass to the capillary bed in the dermis Where the agent may be systemically incorporated into the subject to achieve a therapeutic effect. These methods are limited by the amount of energy that may be applied to the skin without causing discomfort and/or skin damage.

Microneedling technologies have been shown to enhance transdermal delivery of a variety of small agents, such as calcein (˜623 Da), desmopressin (˜1070 Da), diclofenac (˜270 Da), methyl nicotinate (˜40 Da), bischloroekl nitrosourea (˜214 Da), insulin (˜5.8 KDa), bovine serum albumin (˜66.5 KDa) and ovalbumin (˜45 KDa), however until the present disclosure, delivery and/or improved bioavailability of large agents, particularly those of 100 KDa or greater, remained problematic.

### Transdermal Delivery of Large Agents

Transdermal delivery of large agents (e.g., large molecules) is recognized to pose a major challenge. The present Applicant has demonstrated (see, e.g., U.S. Patent Application No. 62/774,677, U.S. Patent Application No. 62/789,407, and U.S. Patent Application No. 62/808,274) that microneedling, and in particular microneedle skin preconditioning using relatively low microneedle density and/or relatively small microneedle puncture size (e.g. puncture size per microneedle), has surprising impact(s) and/or effect(s) on transdermal administration of large agents (e.g., botulinum toxin). Such demonstration was surprising in light of the state of the art, which included, for example, a study of the use of solid microneedles for delivery of four hydrophilic peptides of low molecular weight tetrapeptide-3 (456.6 Da); hexapeptide (498.6 Da); acetyl hexapeptide-3 (889 Da); and oxytocin (1007.2 Da), as well as L-carnitine (161.2 Da). This study had shown that, while microneedle pretreatment significantly enhanced the penetration of each of the tested peptides, the skin permeation of the peptides depended on their molecular weight and decreased as the molecular weight increased. Zhang, S., et al., “Enhanced delivery of hydrophilic peptides in vitro by transdermal microneedle pretreatment.” Acta Pharmaceutica Sinica B. 4(1):100-104 (2014).

Furthermore, when sandpaper abrasion, tape stripping, and a single puncture hypodermic needle model of MSC were compared in a study of the effect of molecular size of larger FITC (fluorescein isothiocyanate) conjugated molecules on transdermal delivery, it was found that for all methods, as well as when tested on untreated skin, transdermal drug delivery was again shown to be reduced as the size of the test molecules increased (4.3, 9.6 and 42.0 KDa FITC conjugates). Tape stripping was the most effective technique, while sandpaper abrasion was found to be the most skin damaging. Wu, X., et al., “Effects of pretreatment of needle puncture and sandpaper abrasion on the in vitro skin permeation of fluorescein isothiocyanate (FITC)-dextran.” International Journal of Pharmaceutics. 316:102-108 (2006).

Other studies attempted delivery of even larger molecules: Cascade Blue (CB, Mw 538), Dextran-Cascade Blue (DCB, Mw 10 kDa), and FITC coupled Dextran (FITC-Dex, Mw 72 kDa), In that study, microneedles of varying lengths (300, 550, 700 or 900 μm) were used to puncture dermatomed human skin and the diffusion of each of the aforementioned compounds was assessed. While transportation of each of the compounds was seen with all but the 300 μm microneedle array, degradation of the DCB and FITC-Dex was observed.

Prior to work of the present Applicant, the understanding in the art was that, as molecular size increases, transdermal penetration using MSC (“microneedle skin conditioning”) decreases, to the point where it is de minimis and even non-existent. Even in those cases where some de minimis penetration was observed, the larger molecules were observed to become degraded and biologically inactive. Work by the present Applicant (including, for example, as described in International Patent Application No. PCT/US17/53333; published as WO2018/093465) has demonstrated that various advantages are achieved by combining microneedling technologies with emulsion technologies for transdermal delivery of large agents of interest; in some embodiments, these technologies have shown particularly surprising enhancements can be achieved for transdermal delivery of large molecular structures without the use of mechanical or chemical permeation enhancers. For example, in some embodiments, these technologies have achieved transdermal delivery of botulinum which, at approximately 150 KDa, is more than twice the size of FITC-Dex.

The present disclosure provides certain technologies that have surprising properties and/or achieve unexpected benefits useful in the administration (e.g., via transdermal delivery) of large molecular agents, and particularly of botulinum toxin.

Those skilled in the art will be aware of a variety of protein agents that have been approved for therapeutic use by a relevant regulatory authority. For example, the United. States Food and Drug Administration maintains a list of approved biologic therapeutics; organized by year of approval, that can be found at: www.fdagov/biologicsbloodvaccines/developmentapprovalprocess/biologicalapprovalsbyyear/ucm547553.htm. Those skilled in the art, reading the present disclosure, will appreciate that its teachings may be applicable to any of a variety of such agents; of particular interest are those intended and/or formulated for topical administration, including for example, those that may be undergoing or may have undergone clinical testing (e.g., as may be approved or undergoing approval by the United States Food and Drug Administration or its equivalent in another jurisdiction, or as may be included in a clinical trial, such as may be listed at www.clinicaltrials.gov and/or in records of Institutional Review Boards, or equivalents thereof, at one or more clinical sites).

Those skilled in the art, reading the present disclosure, will appreciate that, in some embodiments, a large agent with respect to which its teachings are relevant may be or comprise an antibody agent.

In some embodiments, an antibody agent may be suitable for treating a dermatological condition. In some embodiments an antibody agent may be a fusion protein. In some embodiments an antibody agent may be conjugated to another moiety. In some embodiments, an antibody agent may be conjugated to polyethylene glycol. In some embodiments, an antibody may be multispecific (e.g., bi-specific) and able to attach to two or more different target antigens or epitopes.

In some embodiments, an antibody agent targets TNFα (e.g., includes epitope binding elements found in an anti-TNFα antibody such as infliximab, adalimumab, golimumab, etanercept, etanercept-szzs, and/or certolizumab pegol). In some embodiments, an antibody agent targets CD2 (e.g., includes epitope binding elements found in an anti-CD2 antibody such as siplizumab). In some embodiments, an antibody agent targets CD4 (e.g., includes epitope binding elements found in an anti-CD4 antibody such as zanolimumab).

In some embodiments, an antibody agent targets IL-12 (e.g., includes epitope binding elements found in an anti-IL-12 antibody such as briakinumab). In some embodiments, an antibody agent targets IL-17 (e.g., includes epitope binding elements found in an anti-IL-17 antibody such as secukinumab and/or brodalumab). In some embodiments, an antibody agent targets IL-22 (e.g., includes epitope binding elements found in an anti-IL-22 antibody such as fezakinurnab), in some embodiments, an antibody agent targets IL-23 (e.g., includes epitope binding elements found in ustekinumab and/or guselkumab).

Those skilled in the art, reading the present disclosure, will appreciate that, in some embodiments, a large agent with respect to which its teachings are relevant may be or comprise prophylactic agent such as a vaccine. In some embodiments, vaccines may comprise isolated proteins or peptides, inactivated organisms and viruses, dead organisms and virus, genetically altered organisms or viruses, and cell extracts. In some embodiments, prophylactic agents may be combined with interleukins, interferon, cytokines, and adjuvants such as cholera toxin, alum, Freund's adjuvant, etc. In some embodiments, prophylactic agents may include antigens of such bacterial organisms as Streptococcus pnuemoniae, Haemophilus influenzae, Staphylococcus aureus, Streptococcus pyrogenes, Corynebacterium diphtheriae, Listeria monocytogenes, Bacillus anthracis, Clostridium tetani, Clostridium botulinum, Clostridium perfringens, Neisseria meningitidis, Neisseria gonorrhoeae, Streptococcus mutans, Pseudomonas aeruginosa, Salmonella typhi, Haemophilus parainfluenzae, Bordetella pertussis, Francisella tularensis, Yersinia pestis, Vibrio cholerae, Legionella pneumophila, Mycobacterium tuberculosis, Mycobacterium leprae, Treponema pallidum, Leptospirosis interrogans, Borrelia burgdorleri, Campylobacter jejuni, and the like; antigens of such viruses as smallpox, influenza A and B, respiratory syncytial virus, parainfluenza, measles, HIV, varicella-zoster, herpes simplex 1 and 2, cytomegalovirus, Epstein-Barr virus, rotavirus, rhinovirus, adenovirus, papillomavirus, poliovirus, mumps, rabies, rubella, coxsackieviruses, equine encephalitis, Japanese encephalitis, yellow fever, Rift Valley fever, hepatitis A, B, C, D, and E virus, and the like; antigens of fungal, protozoan; and parasitic organisms such as Cryptococcus neoformans, Hisoplasma capsulatum, Candida albicans, Candida tropicalis, Nocardia asteroides, Rickettsia rickets/i, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis, Schistosoma mansoni, and the like. In some embodiments, these antigens may be in the form of whole killed organisms, peptides, proteins, glycoproteins, carbohydrates, or combinations thereof.

Those skilled in the art will recognize that the preceding paragraphs provide an exemplary, not comprehensive, list of agents that can be delivered using technologies in accordance with the present invention; and will appreciate the applicability of provided teachings to administration of other agents.

### Botulinum Toxin Therapies

Those skilled in the art are aware that Botulinum toxin is a potent and effective inhibitor of acetylcholine release. Acetylcholine is a neurotransmitter that is active, for example, at neuromuscular junctions, and at certain other synapses (e.g., within the central nervous system and in ganglia, particularly within the visceral motor system). Botulinum toxin is useful to in the treatment and/or prevention of a variety of diseases, disorders, and conditions, particularly including those associated with acetylcholine release and/or activity.

In nature, Botulinum toxin is produced by Clostridium botulinum, a gram-positive anaerobic bacterium. Botulinum toxins are classified into one of seven antigenically distinct but structurally similar classes: A, B, C (C1 or C2), D, E, F, and G All seven are understood to inactivate (by enzymatic cleavage) a protein required for the docking and fusion process involved in acetylcholine release.

Botulinum toxin is naturally produced as a single polypeptide (about 150 kD) that forms an intra-peptide disulfide bond, and becomes cleaved to generate the di-peptide toxin, whose chains are connected to one another via the disulfide bond. The light chain (approximately 50 kD) has endopeptidase activity; the heavy chain binds to presynaptic receptors and also promotes light chain translocation across the endosomal membrane.

Botulinum toxins are released by the Clostridium bacterium as complexes comprising a 150 kDa botulinum toxin protein molecule along with associated non-toxin proteins. Thus, a BTX-A complex can be produced by Clostridium bacterium as 900 kDa, 500 kDa and 360 kDa forms. Botulinum toxin types B and C₁ are apparently produced as only a 500 kDa complex. Botulinum toxin type D is produced as both 300 kDa and 500 kDa complexes. Finally, botulinum toxin types E and F are produced as only approximately 300 kDa complexes.

A variety of Botulinum toxin therapeutics have been approved by the United States Food and Drug Administration, including for example:
a) AbobotulinumtoxinA (marketed as Dysport®, which is approved for use in achalasia, blepharospasm associated with dystonia, cervical dystonia (spasmodic torticollis), chronic anal fissures, detrusor overactivity (detrusor hyperreflexia) or detrusor-sphincter dyssynergia due to spinal cord injury or disease, hand dystonia, hand tremor, hemifacial spasm, hyperhidrosis including gustatory sweating (Frey's Syndrome), moderate-to-severe glabellar lines in adults, oromandibular dystonia, sialorrhea, spasmodic dysphonia (laryngeal dystonia), stasticity associated with cerebral palsy or multiple sclerosis or neuromyelitis optica or stroke or other injury disease or tumor of the brain or spinal cord, strabismus, tongue dystonia, torsion dystonia, and upper and lower limb spasticity, including lower limb spasticity in children aged 2 years or older), voice tremor, etc;
b) IncobotulinumtoxinA (marketed as Xeomin®, which is approved for use in blepharospasm, cervical dystonia, chronic sialorrhea, moderate to severe glabellar lines, spasticity associated with cerebral palsy or multiple sclerosis or neuromyelitis optica or stroke or other injury disease or tumor of the brain or spinal cord, etc);
c) OnabotulinumtoxinA (marketed as Botox® which is approved for use in achalasia, blepharospasm, cervical dystonia, chronic anal fissures, chronic migraine, neurogenic detrusor overactivity, hand dystonia, hand tremor, hemifacial spasm, hyperhidrosis including gustatory sweating (Frey's Syndrome), oromandibular dystonia, overactive bladder, sialorrhea, spasmodic sydphonia (laryngeal dystonia), severe primary axillary hyperhidrosis, spasticity associated with cerebral palsy or multiple sclerosis or neuromyelitis optica or stroke or other injury disease or tumor of the brain or spinal cord, strabismus, tongue dystonia, torsion dystonia, voice tremor, etc.; and Botox Cosmetic®, which is approved for use with moderate to severe lateral canthal lines, known as crow's feet and moderate to severe glabellar lines),
d) PrabotulinumtoxinA-xcfs (marketed as Jeuveau™ and approved for use in temporary improvement in the appearance of moderate to severe glabellar lines associated with corrugator and/or procerus muscle activity in adults); and
e) RimabotulinumtoxinB (marketed as Myobloc®, which is approved for use in cervical dystonia, detrusor overactivity (detrusor hyperreflexia), sialorrhea, spasticity associated with cerebral palsy or multiple sclerosis or neuromyelitis optica or stroke or other injury disease or tumor of the brain or spinal cord, etc).

Those skilled in the art are aware of standard and/or approved administration regimens for such commercially available botulinum toxin compositions and, upon reading the present disclosure, will appreciate how and to what extent relevant such compositions and/or regimens may be utilized together with microneedling technologies (e.g., specifically with MSC), as described herein. Alternatively or additionally, those skilled in the art, reading the present disclosure, will appreciate the extent to which such approved products and/or regimens may be considered to be an appropriate reference against which timing of peak effect and/or duration of response may be assessed as described herein.

Botulinum toxin use is considered to be cosmetic when associated with improving a feature of appearance, or in the absence of physiological functional impairment expected to be improved by administration of the toxin; use is considered to be therapeutic (in the presence of such functional impairment), and is considered to be prophylactic if administered prior to development of significant symptoms or characteristics of a relevant cosmetic feature of physiological functional impairment.

For treatment of wrinkles in particular, the United. States Food and Drug Administration (FDA) has provided a draft Guidance to Industry recommending that “Measurements at maximum contraction should be used to assess the efficacy of botulinum toxin drug products to demonstrate the paralytic effect” and that: “Success should be defined as . . . a two-grade improvement from the baseline, on both the [investigator's assessment] and the [subject's self-assessment] scales concurrently, to ensure clinical significance.” See www.fda.gov/downioads/Drugs/GuidanceComplianeeRegulatoryinformation/Guidances/UCM407983.pdf.

Botulinum toxin is a complex protein, requiring three regions or functional moieties to be intact in order for the protein to be biologically active. Thus, damage to any one of the three regions of the protein make the protein inactive biologically. Per Johnson, E., et al., “Botulinum toxin is very susceptible to denaturation due to surface denaturation, heat, and alkaline conditions.” US patent Publication No, 5512547. Many traditional microneedling conditions (e.g., those described by Wu) would be expected to risk a significant level of degradation and inactivation of the botulinum. Findings documented in the present disclosure—e.g., that nanoemulsion composition administration, in combination with microneedle skin conditioning, can delay peak effect and/or extend duration of response are particularly surprising in light of this known liability of botulinum toxin.

Those skilled in the art, reading the present disclosure, will appreciate that its teachings may be applicable to administration of botulinum toxin polypeptide and/or botulinum toxin complexes, and/or to any portion or fragment or variant of a botulinum toxin protein or complex that retains relevant activity.

In some embodiments, a botulinum toxin utilized in accordance with the present disclosure may be selected from the group consisting of type A, type Ab, type ref, type B, type Bf, type C1, type C2, type D, type E, type F, and type G; mutants thereof; variants thereof; fragments thereof; characteristic portions thereof; and/or fusions thereof. In some embodiments, botulinum toxin may be a variant toxin, for example having one or more structural variations relative to a reference (e.g., wild type) toxin (or relevant fragment thereof). In some particular embodiments, a variant toxin may have a biologically active life that is longer or shorter than that of an appropriate, comparable reference form (e.g., a wild type form), In some embodiments, botulinum toxin is present as any of the subtypes described in Sakaguchi, 1982, Pharmacol. Ther., 19:165; and/or Smith et al, 2005, Infect. Immun., 73:5450; both of which are incorporated herein by reference.

In some embodiments, a botulinum toxin provided and/or utilized in accordance with the present invention may be or comprise one or more botulinum products that have been approved or are in development, such as, for example AbobotulinumtoxinA, DaxibotulinumtoxinA, Hengli, IncobotulinumtoxinA, Medy-Tox, Neuronox, NT-201, OnabotulinumtoxinA, PrabotulinurntoxinA-xcfs, PurTox, RimabotulinumtoxinB, etc.

Some embodiments of the present invention contemplate a pharmaceutical composition comprising a stabilized botulinum toxin for transdermal delivery into a human patient. The botulinum toxin can be selected from the group consisting of botulinum toxin types A. B, C1, D, E, F and G, an isolated and/or purified (i.e. about 150 kDa) botulinum toxin, as well as a native or recombinantly made botulinum toxin. In some embodiments, a composition can comprise and/or deliver a unit amount of botulinum toxin that may be, for example, between about 1 unit to about 100,000 units, and/or can comprise and/or deliver an amount of botulinum toxin sufficient to achieve a therapeutic effect lasting between about 1 month and about 5 years.

### Duration of Response and Peak Effect

As described in a recent review, “Time to onset of response and duration of response are key measures of botulinum toxin efficacy that have a considerable influence on patient satisfaction”. See Nestor et al. Aesthetic Surg J. 37:S20, 2017. This review also reports that “In general, some patients are aware of an improvement in wrinkles within 1 day of treatment, and return of muscle function generally seems to occur 3 to 6 months after treatment. Patients who have had multiple treatment sessions may find that the duration of effect becomes longer, thus lengthening the interval between injections”, citing Chauhan et al J Maxillofac Oral Surg. 12(2):173, 2013; Helsel et al. J Drugs Dermatol. 2013; 12(12):1356-1362; Jaspers et al Int J Oral Maxillofac Surg. 2011; 40(2):127-133; Michaels et al Aesthet Surg J. 2012; 32(1):96-102; Nestor & Ablon J Drugs Dermatol. 2011; 10(10):1148-1157; Nestor Ablon J Clin Aesthet Dermatol. 2011; 4(9):43-49; Rzany et al. 0.1 Drugs Dermatol. 2013; 12(1):80-84; Schlessinger et al, Dermatol Surg. 2011; 37(10):1434-1442; Yu et al Arch Facial Plast Surg. 2012; 14(3):198-204; Small Am Fam Physician. 2014; 90(3):168-175.

Such multiple treatments may be required to achieve the longer reported duration of effects, as many studies conclude that relevant clinical effects have declined significantly earlier. For example, leading botulinum researcher Carruthers has described that. “In most individuals the clinical effects of botulinum toxin A begin to appear at 1-2 days, peak in 1-4 weeks and gradually decline after 3-4 months,” (Carruthers J, and Carruthers A; Using Botulinum Toxic Cosmetically, A Practical Guide 2011 Inform Healthcare, London, UK.), Furthermore, a controlled clinical trial of the treatment of Crow's Feet wrinkles with injectable botulinum found that the median duration of action was approximately 4.5 months for those that were responders at Day 30 of the clinical trial (Carruthers A, et al. “Efficacy and Safety of OnabotulinumtoxinA for the Treatment of Crows Feet Lines: A Multicenter, Randomized, Controlled Trial” Dermatol Surg 2014; 40:1181-1190). Moreover, the package insert for Botox™ itself states that the duration of effect for the treatment of glabellar lines with injectable botulinum is approximately 3 to 4 months (Botox Cosmetic Package Insert).

While many in the community report an expectation that rapid onset of response is desirable, for example by reporting that “Patients want the effect of treatment to be visible as soon as possible after the procedure,” (see Nestor et al. Aesthetic Surg J. 37:S20, 2017), the present disclosure appreciates that too-fast onset can have disadvantages including, for example, increasing risk of a “frozen face” effect and/or engendering undesirable social commentary or awkwardness if an immediate significant change in facial features is observable by others.

Thus, among other things, the present disclosure identifies the source of a problem in certain available approaches to botulinum toxin therapy in that they are designed to achieve a rapid onset of effect (typically within a day or so—e.g., about 1-3 days—of administration) and/or rapid peak effect (typically within a month of less of administration). The present disclosure appreciates that it may be desirable, particularly for treatments with a visible effect (including cosmetic treatments), to design and/or administer botulinum therapy with a delayed onset and/or peak effect. In some embodiments, such onset or peak effect is assessed for a (e.g., each) single dose; in some embodiments, such onset or peak effect is assessed for the last of a plurality of doses in a regimen.

Those skilled in the art will appreciate that, where multiple administrations have occurred, time to peak effect for a later dose administered to a particular individual may be shorter than was time to peak effect for an earlier dose given to the same individual, due to the presence of an already weakened muscle. Those skilled in the art are familiar with expected variations in time to peak effect for administrations to a given subject and therefore will be able to assess and appreciate a relevant impact as described herein, relative to a comparable dose (e.g., a dose administered to a subject who has comparably received prior dose(s)).

In some embodiments, provided botulinum toxin therapy regimens achieve a peak effect (e.g., for one or more doses within the regimen) later than one month and, in some embodiments later than about 2, about 3, about 4, about 5, about 6, about 7, about 8, about 9, about 10, about 11 months, or even later than a year of more, after administration.

Among other things, the present disclosure provides an insight that delayed peak effect as described herein can have certain advantages, particularly in certain patient populations. For example, many subjects, particularly those receiving cosmetic or otherwise visually apparent botulinum toxin treatment(s) (e.g., reduction of wrinkles, etc) may prefer relatively slow onset and/or rate of development of effect, either or both of which can be embodied in and/or represented by delayed peak effect, so that the fact of their having received the treatment may not be immediately apparent or startling to onlookers. In some embodiments, a delayed peak effect regimen as described herein permits sufficiently gradual onset of action that onlookers may not appreciate that the subject has received a botulinum toxin therapy.

Alternatively or additionally, the present disclosure provides an insight that delayed peak effect as described herein may present a reduced risk of development of “frozen face” or another undesirable potential side effect of certain other botulinum toxin therapies.

Still further, the present disclosure documents a surprising effect of certain botulinum toxin therapies (e.g., delayed peak effect therapies) as described here in that they demonstrate a median duration of response that is greater than that reported for various approved botulinum toxin therapies.

Without wishing to be bound by any particular theory, the present disclosure observes that the delayed peak effect documented herein reveals altered pharmacokinetics and/or pharmacodynamics achieved by provided therapies as compared with certain other botulinum toxin therapies (including, for example, injectable therapies). Those skilled in the art, reading the present disclosure will appreciate that such alterations in pharmacokinetics and/or pharmacodynamics likely also underlie the observed extended duration of effect described herein.

As has already been noted herein, current commercially available botulinum toxin products have a median duration of response of approximately 3-4 months (e.g., as has been reported for treatment of wrinkles according to approved regimens), and typically have a maximum duration of response that is less than 6 months.

The present disclosure documents that provided botulinum toxin therapies, e.g., that involve administering botulinum toxin in combination with microneedle skin conditioning, are characterized by a median duration of effect (e.g., for a single dose of botulinum toxin) that exceeds 6 months. In some embodiments, provided such therapies are characterized by a median duration of response that exceeds 6 months, 7 months, 8 months, 9 months, 10 months, 11 months, or one year or more.

Thus, in some embodiments, provided botulinum toxin therapies (e.g., that involve administering botulinum toxin in combination with microneedle skin conditioning) involve administering two or more individual doses that are separated from one another by a period of time that is greater than about 6 months, 7 months, 8 months, 9 months, 10 months, 11 months, or one year or more. In some embodiments, different pairs of doses within a provided regimen are separated from one another by different such periods of time; in some embodiments, some or all pairs may be separated from one another by the same such period of time.

Those skilled in the art are aware that increasing magnitude of an administered dose can sometimes extend duration of effect for certain therapies. The extended duration of effect provided herein, however, cannot be attributed merely to change in magnitude of dose. Those skilled in the art are aware that increasing dose extends duration of effect without impacting timing of peak effect. For example, studies have demonstrated that increasing the dose of injected botulinum over a three-fold range increases median duration of effect; such dose increase, however, has no effect on timing of peak effect. That is, peak effect for all doses was achieved peak effects within 4 weeks of injection, consistent with other studies of injected botulinum (Ascher B. et al, “Efficacy and Safety of Botulinum Toxin Type A in the Treatment of Lateral Crow's Feet: Double-Blind, Placebo-Controlled, Dose-Ranging Study” Dematol Surg, 2009; 35: 1478-1486).

By marked contrast, as described herein, provided therapies e.g, that combine certain topical botulinum treatments with MSP) both extend duration of effect and also result in a delayed peak effect (i.e., a peak effect much later than 4 weeks), Without wishing to be bound by any particular theory, as noted above, it is believed that these combined effects reflect altered pharmacokinetics and/or pharmacodynamics achieved by technologies provided herein.

Thus, the present disclosure provides technologies for both delaying peak effect and also extending duration of response for an administered large agent e.g., botulinum toxin), for example by combining topical administration (for example of an emulsion formulation such as a nanoemulsion formulation) with MSC, e.g., as described and/or exemplified herein.

### Microneedling

In some embodiments, microneedling (e.g., microneedle skin conditioning) in accordance with the present disclosure is performed with microneedle (MN) arrays that are or share features with minimally invasive systems. In some embodiments, microneedling technologies useful in accordance with the present disclosure avoid and/or overcome one or more disadvantage(s) commonly associated with use of hypodermic and/or subcutaneous needles, as well as improve patient comfort and compliance. Such disadvantages include, for example, potential for needle tip misplacement with a hypodermic needle because a health professional cannot visualize where exactly the needle is going; such needle misplacement can be particularly problematic for administration of botulinum toxin as, for example, it may result in adverse reactions such as a drooping eyelid (“ptosis”) when botulinum toxin is injected incorrectly in the face. Microneedling technologies are less prone to such a problem. Other advantages of microneedling technologies include that they may not cause bleeding, minimize introduction of pathogens through microneedle-produced holes, and/or may eliminate transdermal dosing variability. Still other advantages include the possibility of self-administration, reduce risk of accidental needle stick injuries, reduce risk of transmitting infection, and ease of disposal. In some embodiments, microneedles are multiple microscopic projections assembled on one side of a support, such as a patch or a device (e.g., stamp, roller, array, applicator, pen).

In some embodiments, microneedles for use in accordance with the present disclosure may be designed and/or constructed in arrays, which may, for example, improve skin contact and/or facilitate penetration into the skin. In some embodiments, utilized microneedle s are of suitable length, width, and shape to minimize contact with nerves when inserted into the skin, while still creating efficient pathways for drug delivery. Alkilani, A. Z., et al., “Transdermal drug delivery: Innovative pharmaceutical developments based on disruption of the barrier properties of the stratum corneum.” Pharmaceutics, 7:438-470 (2015).

In some embodiments, a suitable microneedle may be solid, coated, porous, dissolvable, hollow, or hydrogel microneedle. Solid microneedles create microholes in the skin, thereby increasing transport of a drug formulation (e.g., “poke and patch” methods). Coated microneedles allow for rapid dissolution of a coated drug into the skin (e.g., “coat and poke” methods). Dissolvable microneedles allow for rapid and/or controlled release of a drug incorporated within the microneedles. Hollow microneedles may be used to puncture the skin and enable release of a composition following active infusion or diffusion of a formulation through a microneedle's bores (e.g., “poke and flow” methods”).

In the case of dissolvable microneedles, microneedles can act as a drug depot, holding a drug composition until released by dissolution in the case of dissolvable microneedles or swelling in the case of hydrogel microneedles (e.g., “poke and release” methods). However, as already described herein, in many embodiments, the large agent is not delivered by injection via one or more microneedles. That is, in many embodiments, any microneedle utilized in accordance with such embodiments is not coated, loaded, or fabricated with the large agent in any way that would achieve delivery of the large agent.

Alternatively or additionally, in some embodiments, as described herein, a microneedles, utilized in accordance with the present disclosure (whether in MSC or otherwise), may comprise and/or deliver a large agent, if the large agent is formulated in a macro- or nano-emulsion composition as described herein. Thus, as will be appreciated by those skilled in the art reading the specification described herein, treatment of skin with microneedle(s) that deliver the large agent (e.g., by injection through a microneedle, by the release of a microneedle coating or by the release from a dissolving microneedle) is not microneedle skin conditioning.

In some embodiments, a microneedle has a diameter which is consistent throughout the microneedle's length. In some embodiments, the diameter of a microneedle is greatest at the microneedle's base end. In some embodiments, a microneedle tapers to a point at the end distal to the microneedle's base. In some embodiments, a microneedle may be solid. In some embodiments, a microneedle may be hollow. In some embodiments a microneedle may be tubular. In some embodiments, a microneedle may be sealed on one end. In some embodiments, a microneedle is part of an array of microneedles.

In some embodiments, a microneedle may have a length of between about 1 μm to about 4; 000 μm. In some embodiments, a microneedle may have a length of between about 1 μm to about 2,000 μm. In some embodiments, a microneedle may have a length of between about 50 μm to about 400 μm. In some embodiments, a microneedle may have a length of between about 50 μm to about 500 μm. In some embodiments, a microneedle may have a length of between about 50 μm to about 600 μm. In some embodiments, a microneedle may have a length of between about 50 μm to about 700 μm. In some embodiments, a microneedle may have a length of between about 50 μm to about 800 μm. In some embodiments, a microneedle may have a length of between about 800 μm to about 1500 μm. In some embodiments, a microneedle may have a length of less than about 1400 μm. In some embodiments, a microneedle may have a length of less than about 1100 μm. In some embodiments, a microneedle may have a length of less than about 1000 μm. In some embodiments, a microneedle may have a length of less than about 800 μm. In some embodiments, a microneedle may have a length between about 100 μm and about 800 μm.

In some embodiments, microneedling as described herein comprises applying to skin a plurality of microneedles (e.g., a microneedle array) of common length; in some embodiments, microneedling as described herein comprises applying to skin a plurality of microneedles (e.g., a microneedle array) of different lengths.

Microneedles of various lengths may be used in the microneedling technologies described herein. In some embodiments, the length of the microneedles used in MSC as described herein is adjusted based on skin thickness of the treatment site.

In some embodiments, a microneedle or microneedle array comprises microneedles of about 25, about 50, about 100, about 150, about 200, about 250, about 300 about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, or about 1500 μm length.

In some embodiments, a microneedle or microneedle array comprises a plurality of needles some embodiments, a microneedle or microneedle array comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 200, 300, 400, 500, 600, 700, 800, 900, 1000, 1500, 2000 microneedles or more/cm2.

Microneedles of any shape may be used in the microneedling technologies described herein. In some embodiments, microneedles may have a circular cross-section. In some embodiments, microneedles may have a triangular cross-section. In some embodiments, microneedles may have a rectangular cross-section. In some embodiments, microneedles may have a square cross-section. In some embodiments, microneedles may have a quadrangular cross-section. In some embodiments, microneedles may have a pentagular cross-section. In some embodiments, microneedles may have a hexangular cross-section. In some embodiments, microneedles may have a septangular cross-section. In some embodiments, microneedles may have an octangular cross-section. In some embodiments, microneedles may have a nonangular cross-section. In some embodiments, microneedles may have a decangular cross-section.

Microneedles of various cross-sectional areas may be used in the microneedling technologies described herein. The cross-sectional area of each microneedle in the MN array used for MSC (“microneedle skin conditioning”) as described herein, may in turn define the microneedle puncture size (e.g., puncture size per microneedle) of the MN array used for MSC. In some embodiments, microneedle puncture size may be in the range of about 100 to about 60,000 μm²/microneedle. In some embodiments, microneedle puncture size may be in the range of about 100 to about 30,000 μm²/microneedle.

In some embodiments, a microneedle or microneedle array comprises needles with a plurality of microneedle puncture sizes. In some embodiments, a microneedle or microneedle array comprises needles with at least 2 different microneedle puncture sizes. In some embodiments, a microneedle or microneedle array comprises needles with at least 3 different microneedle puncture sizes. In some embodiments, a microneedle or microneedle array comprises needles with at least 4 different microneedle puncture sizes. In some embodiments, a microneedle or microneedle array comprises needles with at least 0.5 different microneedle puncture sizes. In some embodiments, a microneedle or microneedle array comprises needles with at most 10 different microneedle puncture sizes. In some embodiments, a microneedle or microneedle array comprises needles with at least 11 different microneedle puncture sizes. In some embodiments, a microneedle or microneedle array comprises needles with at least 12 different microneedle puncture sizes. In some embodiments, a microneedle or microneedle array comprises needles with at most 1 microneedle puncture size.

In some embodiments, a microneedle or microneedle array comprising microneedles of various microneedle puncture sizes may be used in the microneedling technologies described herein. In some embodiments, a microneedle or microneedle array comprises microneedles with microneedle puncture size of about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2500, about 3000, about 3500, about 4000, about 4500, about 5000, about 5500, about 6000, about 6500, about 7000, about 7500, about 8000, about 8500, about 9000, about 9500, about 10000, about 10500, about 11000, about 11500, or about 12000 μm²/microneedle. In some embodiments, a microneedle or microneedle array comprises microneedles with microneedle puncture size of less than about 13000, less than about 14000, less than about 15000, less than about 20000, less than about 25000, less than about 30000, less than about 35000, less than about 40000, less than about 45000, less than about 50000, less than about 55000, or less than about 60000 μm²/microneedle.

In some embodiments, utilized microneedles may be solid; in some embodiments, utilized microneedles may be hollow; in some embodiments, utilized microneedles may be uncoated; in some embodiments, utilized microneedles may be coated (e.g., with a composition that may be or comprise a large agent as described herein).

In some embodiments, MN for use in accordance with the present disclosure may be fabricated from different materials, using technologies including, but not limited to micro-molding processes or lasers. In some embodiments, MN may be manufactured using various types of biocompatible materials including polymers, metal, ceramics, semiconductors, organics, composites, or silicon. Unless they are designed to break off into the skin and dissolve, in some embodiments, microneedles have the mechanical strength to remain intact and to deliver drugs, or collect biological fluid, while being inserted into the skin and/or removed from the skin after insertion. In some embodiments MN are capable of remaining in place for up to a number of days before intact removal. In some embodiments, microneedles may be sterilizable using standard technologies. In some embodiments, MN are biodegradable. In some embodiments, MN comprise a polymeric material. In some embodiments the polymeric material comprises poly-L-lactic acid, poly-glycolic acid, poly-carbonate, poly-lactic-co-glycolic acid (PLEA), polydimethylsiloxane, polyvinylpyrrolidone (PVP), a copolymer of methyl vinyl ether and maleic anhydride, sodium hyaluronate, carboxymethyl cellulose, maltose, dextrin, galactose, starch, gelatin, or a combination thereof.

In some embodiments, MSC as described herein comprises one impression of MN or MN array. In some embodiments, MSC comprises two impressions of MN or MN array. In some embodiments, MSC comprises three impressions of MN or MN array. In some embodiments, MSC comprises four impressions of MN or MN array. In some embodiments, MSC comprises five impressions of MN or MN array. In some embodiments, MSC comprises six impressions of MN or MN array. In some embodiments, MSC comprises seven impressions of MN or MN array. In some embodiments, MSC comprises eight impressions of MN or MN array. In some embodiments, MSC comprises nine impressions of MN or MN array. In some embodiments, MSC comprises ten impressions of MN or MN array. In some embodiments, MSC comprises eleven impressions of MN or MN array. In some embodiments, MSC comprises twelve impressions of MN or MN array. In some embodiments, MSC comprises thirteen impressions of MN or MN array. In some embodiments, MSC comprises fourteen impressions of MN or MN array. In some embodiments, MSC comprises fifteen impressions of MN or MN array. In some embodiments. MSC comprises sixteen impressions of MN or MN array. In some embodiments, MSC comprises seventeen impressions of MN or MN array. In some embodiments, MSC comprises eighteen impressions of MN or MN array. In some embodiments, MSC comprises nineteen impressions of MN or MN array. In some embodiments, MSC comprises twenty impressions of MN or MN array. In some embodiments, the MSC comprises rolling a microneedle or microneedle array over the skin one or more times. In some embodiments, an MN array is rotated between impressions. In some embodiments an MN array is not rotated between impressions. In some embodiments impressions are made on the same site. In some embodiments impressions are made on overlapping sites. In some embodiments, impressions are made on different sites. In some embodiments, impressions are made by stamping of a MN array. In some embodiments, impressions are made by rolling a microneedle roller over a site one or more times. In accordance with established MN practices, in some embodiments, the MN array skin impressions last under one second or, alternatively, in some embodiments, they last over one second and may, for example, last for 30 seconds or more, 60 seconds or more, two minutes or more, five minutes or more, ten minutes or more, thirty minutes or more, etc.

In some embodiments, the present disclosure appreciates that as aggregate surface area of skin that is punctured by the microneedles decreases, bioavailability of a large agent in an emulsion applied to the skin increases. See, for example, U.S. Patent Application No. 62/789,407. Thus, in some embodiments, relatively fewer impressions may be preferred. In some embodiments, fewer microneedle array impressions may be preferred when a large agent that is being administered in conjunction with microneedle skin conditioning is in a topical formulation that is not (or does not comprise) an emulsion (e.g., an emulsion containing the agent). In some embodiments, shorter microneedle lengths may be preferred. In some embodiments, relatively shorter microneedle lengths may be preferred when a large agent that is being administered in conjunction with microneedle skin conditioning is in a topical formulation that is not (or does not comprise) an emulsion (e.g., an emulsion containing the agent).

Furthermore, in some embodiments, the present disclosure appreciates that application of relatively reduced amount (e.g., volume and/or dose) of product containing a biologically active agent (e.g. large agent) in conjunction with MSC, can achieve greater biological effects. See, for example, U.S. Patent Application No. 62/808,274. Thus, in some embodiments, relatively reduced product volumes containing active agent (e.g. large agent) may be preferred. In some embodiments, relatively smaller product volumes may be preferred when a large agent that is being administered in conjunction with microneedle skin conditioning is in a topical formulation that is or comprises and emulsion (e.g., a nanoemulsion). In some embodiments, relatively smaller product volumes may be preferred when a large agent that is being administered in conjunction with microneedle skin conditioning is in a topical formulation that is not (or does not comprise) an emulsion (e.g., an emulsion containing the agent). Suitable MN arrays and MSC devices for use in combination with compositions comprising large agents for transdermal delivery of large agents include devices such as those described in e.g., U.S. Pat. Nos. 6,334,856; 6,503,231; 6,908,453; 8,257,324; and 9,144,671.

In some embodiments, MSC of a site is performed before applying (e.g., before a particular application and/or before each application of) a formulation (e.g., comprising an emulsion composition such as a nanoemulsion composition) that comprises and/or delivers a large agent to the site. In some embodiments. MSC of a site is performed after applying such a formulation to the site. In some embodiments, MSC of a site and applying such a formulation to the site occur at substantially the same time.

In some embodiments, a formulation is not injected via one or more microneedles. In some embodiments, a microneedle is part of an array of microneedles. In some embodiments, a microneedle may have a length of between about 1 μm to about 4,000 μm. In some embodiments, a microneedle may have a length of between about 1 μm to about 2,000 μm. In some embodiments, a microneedle may have a length of between about 50 μm to about 400 μm. In some embodiments, a microneedle may have a length of between about 800 μm to about 1500 μm.

### Emulsion Compositions

In some embodiments, the present disclosure provides and/or utilizes emulsion compositions (e.g., that include or are otherwise utilized together with a large agent such as botulinum toxin). In some embodiments, formulations described herein that comprise and/or deliver a large agent may be or comprise emulsions (e.g., an emulsion that includes the large agent)

The present disclosure encompasses the recognition that emulsion technologies can provide stabilization benefits to agents of interest, including to large agents as described herein, and specifically including botulinum toxin and/or antibody agents.

Moreover, the present disclosure appreciates that certain liquid nanoemulsion technologies have been demonstrated to provide remarkable transdermal delivery attributes, even for very large molecules, such as botulinum and/or antibody agents. See, e.g., U.S. Patent Publication No. 2012/0328701, U.S. Patent Publication No. 2012/0328702, 8,318,181, and U.S. Pat. No. 8,658,391, the disclosures of which are herein incorporated by reference in their entireties. These liquid nanoemulsions are far superior to solid nanoparticle drug delivery, particularly transdermal drug delivery wherein, as noted by Gomaa, the solid nanoparticles cannot penetrate the skin but merely accumulate in the hair follicles These liquid nanoemulsions are also stable for at least 34 months, making them a commercially viable from this perspective as well.

The present disclosure provides certain technologies in which administration of an emulsion composition, together with microneedling, achieves surprising results such as, for example, delayed peak effect and/or extended duration of response.

Particular emulsion compositions of interest may be or comprise water-in-oil or oil-in-water emulsions (e.g., liquid emulsions comprising an oil phase in which aqueous droplets are dispersed, or comprising an aqueous phase in which oil droplets are dispersed).

In some embodiments, an emulsion may be or comprise a macroemulsion, e.g., characterized by droplet sizes within a range of about 300 nm to about 5,000 μm in diameter. In some embodiments, an emulsion may be or comprise a nanoemulsion, e.g., characterized by droplet sizes within a range of about 1 nm to about 300 nm in diameter.

In some embodiments, use of a nanoemulsion may achieve more extensive and/or deeper transdermal penetration which may, for example, be attributable at least in part to the nanoemulsion itself (e.g., as compared with that achieved with an alternative composition, including, in some embodiments, with a macroemulsion).

In some embodiments, a useful emulsion may be characterized by one or more of a ratio of aqueous dispersion media to oil ranging between about 0.01:1 to about 20:1; oil-to-surfactant ratio in the range of between about 0.1 to about 40 and/or zeta potential in the range of between about −80 mV to about +80 mV.

In some embodiments, provided emulsion (e.g., nanoemulsion) compositions comprise oil and surfactant at a ratio within between about 0.1:1 to about 2:1. In some embodiments, provided emulsion compositions comprise oil and surfactant at a ratio of about 0.1:1 to about 1:1. In some embodiments, provided emulsion compositions comprise oil and surfactant at a ratio of about 05:1 to about 1:1. In some embodiments, provided emulsion compositions comprise oil and surfactant at a ratio of about 0.5:1 to about 1:1.5. In some embodiments, provided emulsion compositions comprise oil and surfactant at a ratio of about 0.1:1, about 0.15:1, about 0.2:1, about 0.25:1, about 0.3:1, about 0.35:1, about 0.4:1, about 0.45:1, about 0.5:1, about 0.5:1, about 0.55:1, about 0.6:1, about 0.65:1, about 0.7:1, about 0.75:1, about 0.8:1, about 0.85:1, about 0.9:1, about 0.95:1, or about 1:1 In some embodiments, provided emulsion compositions comprise oil and surfactant at a ratio of about 0.67:1.

In some embodiments, the aqueous dispersion medium (e.g., water, buffer, salt solution, etc.) and surfactant are utilized, at a ratio ranging between 0.01 and 20. In some embodiments, the aqueous dispersion medium (e.g., water, buffer, salt solution, etc.) and surfactant are utilized at a ratio ranging between 0.1 and 20. In some embodiments, the aqueous dispersion medium (e.g., water, buffer, salt solution, etc. and surfactant are utilized at a ratio ranging between 0.5 and 10. In some embodiments, the aqueous dispersion medium (e.g., water, buffer, salt solution, etc.) and surfactant are utilized at a ratio ranging between 0.5 and 1. In some embodiments, the ratio of aqueous dispersion medium (e.g., water, buffer, salt solution, etc.) to surfactant is approximately 0.01:1, approximately 0.02:1, approximately 0.03:1, approximately 0.04:1, approximately 0.05:1, approximately 0.06:1, approximately 0.07:1, approximately 0.08:1, approximately 0.0:1, approximately 0.1:1, approximately 0.2:1, approximately 0.3:1, approximately 0.4:1, approximately 0.5:1, approximately 1:1, approximately 2:1, approximately 3:1, approximately 4:1, approximately 5:1, approximately 6:1, approximately 7:1, approximately 8:1, approximately 9:1 or approximately 10:1. In some embodiments, the ratio of surfactant to water is approximately 0.5:1, approximately 1:1, approximately 2:1, approximately 3:1, approximately 4:1, approximately 5:1, approximately 6:1, approximately 7:1, approximately 8:1, approximately 9:1, approximately 10:1, approximately 11:1, approximately 12:1, approximately 13:1, approximately 14:1, approximately 15:1, approximately 16:1, approximately 17:1, approximately 18:1, approximately 19:1, or approximately 20:1. In some embodiments, aqueous dispersion medium (e.g., water, buffer, salt solution, etc.) and surfactant are utilized at a ratio ranging between 0.5 and 2. In some embodiments, the ratio of aqueous dispersion medium (e.g., water, buffer, salt solution, etc.) to surfactant is approximately 0.5:1, approximately 1:1, or approximately 2:1. In some embodiments, the ratio of surfactant to aqueous dispersion medium (e.g., water, buffer, salt solution, etc.) is approximately 0.5:1, approximately 1:1, or approximately 2:1. In some embodiments, the ratio of aqueous dispersion medium (e.g., water, buffer, salt solution, etc.) to surfactant is approximately 1:1. In some embodiments, compositions utilizing such ratios of aqueous dispersion medium (e.g., water, buffer, salt solution, etc.) to surfactant comprise water-in-oil emulsions.

In some embodiments, droplets within nanoemulsion compositions have diameters (e.g., average and/or median diameters) within a range of about 10 nm to about 300 nm, about 10 nm to about 200 nm, about 10 nm to about 150 nm, about 10 nm to about 130 nm, about 10 nm to about 120 nm, about 10 nm to about 115 nm, about 10 nm to about 110 nm, about 10 nm to about 100 nm, or about 10 nm to about 90 nm. In some embodiments, droplets within nanoemulsion compositions have diameters (e.g., average and/or median diameters) within a range of 1 nm to 300 nm, 1 nm to 200 nm, 1 nm to 150 nm, 1 nm to 120 nm, 1 nm to 100 nm, 1 nm to 75 nm, 1 nm to 50 nm, or 1 nm to 25 nm. In some embodiments, droplets within nanoemulsion compositions have diameters (e.g., average and/or median diameters) of 1 nm to 15 nm, 15 nm to 200 nm, 25 nm to 200 nm, 50 nm to 200 nm, or 75 nm to 200 nm.

In some embodiments, a total droplet distribution is encompassed within a specified range of droplet diameter size. In some embodiments, less than 50%, 25%, 10%, 5%, or 1% of a total droplet distribution is outside of a specified range of droplet diameter sizes. In some embodiments, less than 1% of a total droplet distribution is outside of a specified range of droplet diameter sizes.

In some embodiments, a nanoemulsion composition is substantially free of droplets having a diameter larger than 300 nm, 250 nm, 200 nm, 150 nm, 120 nm, 100 nm, 75 nm, 50 nm, or 25 nm. In some embodiments, less than 50%, 25%, 10%, 5%, or 1% of a total droplet distribution have diameters larger than 300 nm, 250 nm, 200 nm, 150 nm, 120 nm, 100 nm, 75 nm, 50 nm, or 25 nm.

In some embodiments, droplets within nanoemulsion compositions have an average droplet size that is under about 300 nm, about 250 nm, about 200 nm, about 150 nm, about 130 nut, about 120 nm, about 115 nm, about 110 nm, about 100 nm, about 90 nm, or about 50 nm. In some embodiments, average droplet size is within a range of about 10 nm and about 300 nm, about 50 nm and about 250, about 60 nm and about 200 nm, about 65 urn and about 150 nm, or about 70 nm and about 130 nut. In some embodiments, average droplet size is about 80 nm and about 110 nm, in some embodiments, average droplet size is about 90 nm and about 100 nm.

In some embodiments, emulsion (e.g., nanoemulsion) droplets have a zeta potential ranging between −80 mV and +80 mV. In some embodiments, emulsion droplets have a zeta potential ranging between −50 mV and +50 mV. In some embodiments, emulsion droplets have a zeta potential ranging between −25 mV and +25 mV. In some embodiments, emulsion droplets have a zeta potential ranging between n −10 mV and +10 mV. In some embodiments, emulsion droplets have a zeta potential of about −80 mV, about −70 mV, about −60 mV, about 50 mV, about −40 mV, about −30 mV, about −25 mV, about −20 mV, about −15 mV, about −10 mV, or about −5 mV. In some embodiments, emulsion droplets have a zeta potential of about +50 mV, about +40 mV, about +30 mV, about +25 mV, about +20 mV, about +15 MV, about +10 mV, or about +5 mV. In some embodiments, emulsion droplets have a zeta potential that is about 0 mV.

Among other things, the present disclosure appreciates that use of an emulsion composition may provide stability to a large agent so that, for example, a relevant agent may remain intact to a greater extent and/or may retain activity to a greater degree and/or for a longer period of time when maintained in an emulsion composition than when maintained under comparable conditions in an otherwise comparable composition.

The present disclosure appreciates that, notwithstanding certain reports in the art that microneedle skin conditioning is helpful in facilitating transdermal delivery of small compounds, transdermal delivery of large agents (e.g., botulinum toxin) can be significantly facilitated through use of an emulsion composition in combination with microneedle skin conditioning as described herein, including to achieve unexpected results such as, for example, delayed peak effect and/or extended duration of response. The present disclosure provides an insight that such technologies are particularly useful in certain contexts (e.g., for the treatment of certain subjects and/or sites thereon—in particular those subjects suffering from particular diseases disorders or conditions and/or site(s) reflective thereof, for which delayed peak effect and/or extended duration of response may be particularly desirable.

[01.57] The present disclosure particularly appreciates that emulsion compositions in combination with microneedling can achieve transdermal delivery that is surprisingly effective in light of reports that microneedle conditioning in combination with encapsulation of even small molecule agents in solid nanoparticles (e.g., 105±2.92 nm) provided for small amounts of penetration only after 6 hours of administration, and no material penetration was observed until 24 hours after administration. For example, Gomaa et al described a study in which a solution of rhodamine dye (molecular weight 479 Da) encapsulated in PLGA nanoparticles was applied to skin that had been preconditioned by microneedling, and skin penetration was assessed. See Gomaa, Y., et al, “Effect of microneedle treatment on the skin permeation of a nanoencapsulated dye.” J Pharm Pharmacol. 2012 November; 64(11): 1592-1602. The data showed that very small amounts of dye began to permeate the skin after 6 hours of continuous application; no significant increase in permeation was observed until skin had been treated continuously for 24 hours. The researchers explained that “there is an emerging consensus that NPs [nanoparticles] cannot usually penetrate the stratum corneum, although they may well deposit in hair follicles.” Thus, prior to the present disclosure, those skilled in the art would expect that use of microneedling technologies with vehicles significantly larger than 105 nm could not effectively deliver even small molecule agents (e.g., rhodamine dye) transdermally; certainly delivery of large agents would have been considered impossible. The present disclosure, however, teaches that microneedling can significantly enhance transdermal delivery of large agents, particularly when utilized in conjunction with emulsion technologies—even including macroemulsion technologies, which involve particles or droplets materially larger than those used by Gomaa and therefore expected to be less able to achieve transdermal delivery.

Among other things, the present disclosure appreciates that provided technologies can enhance transdermal delivery (e.g., of large agents, particularly from macroemulsion compositions), when no other disrupting agent (i.e., no chemical penetration enhancing agent and no other technology that disrupts or punctures skin structure) is utilized. In some embodiments, therefore, provided technologies therefore can achieve effective delivery without inflammation, irritation, and/or allergic reaction that often accompanies use of skin disrupting agents.

Prior studies of transdermal delivery of an agent as large as botulinum toxin (i.e., about 150 kDa) using microneedles have reported that delivery is unsuccessful unless additional treatment is applied to disrupt skin. For example, U.S. Patent Publication No. 2010/0196445 reports that botulinum toxin is not delivered effectively from pre-coated microneedles unless a skin-digesting enzyme is also applied, so that skin structure is disrupted at the site of microneedling.

The present disclosure appreciates, among other things, that provided technologies can achieve transdermal delivery (e.g., of large agents, particularly from microemulsion and nanoemulsion compositions), when no coating or loading of the microneedles is utilized and/or when the microneedles are not designed to be left in the skin. Among other things, as already noted, the present disclosure appreciates that such coating or loading of microneedles might not be commercially viable, at least due to the instability of the coating or material (e.g., large agent such as botulinum toxin) loaded in it. For example, per Johnson, E., et al., “Botulinum toxin is very susceptible to denaturation due to surface denaturation, heat, and alkaline conditions. Lyophilization or freeze-drying of botulinum toxin is the most economically sound and practical method of distributing the product in a form that is stable and readily used by the clinician.” U.S. Pat. No. 5,512,547.

Additionally, as will be appreciated by those skilled in the art reading the specification, technologies described herein have certain advantages including that it is not necessary that microneedles be left in or in association with tissue. For example, those skilled in the art will appreciate that leaving the microneedles in the skin can risk skin irritation, inflammation, allergic reaction, and/or cosmetically undesirable scarring. In contrast to the present invention, technologies such as that described in US Patent Publication No. 2017/0209553 utilize a microneedle array that is loaded with botulinum into the needles and is designed for the microneedles to break off into the skin (per US patent No. 2017/0209553 and 2016/0263362; also see International Patent Publication No. WO/2018/151832).

The present disclosure provides surprisingly effective technologies for transdermal delivery of large agents (e.g., botulinum toxin, antibodies, etc.). In particular, the present disclosure teaches that transdermal delivery of such agents can be significantly enhanced through use of microneedling technologies without any other disrupting strategy. Provided technologies therefore can achieve effective delivery without inflammation, irritation, and/or allergic reaction that often accompanies use of skin disrupting agents. As will be appreciated by those skilled in the art reading the specification, the present disclosure teaches that transdermal delivery of such large agents can be significantly enhanced through use of provided technologies even when the large agent is not loaded into, coated on, and/or fabricated as part of the microneedles. Similarly, as will be appreciated by those skilled in the art reading the specification, the present disclosure teaches that delivery of large agents as described herein can be significantly enhanced through use of provided technologies (and specifically through use of MSC), without leaving microneedles in the skin (e.g., by having them break off and/or otherwise be retained and/or degraded in situ). For example, those skilled in the art will appreciate that provided technologies can avoid problems with the long-term stability of the large agent necessary for a commercially viable product, and can achieve effective delivery without inflammation, irritation, and/or allergic reaction that may result from the skin disrupting agents and/or the microneedles being left in the skin. Indeed, in the examples and elsewhere, the present disclosure explicitly teaches that MSC performed with microneedles that contain no botulinum toxin facilitates transdermal delivery of botulinum toxin from a topical (e.g., cream, ointment) composition, and particularly from a composition comprising a macro- or nano-emulsion.

In some embodiments, the present disclosure teaches that particularly advantageous results are achieved when microneedling technologies are combined with emulsion compositions. In some embodiments, microneedling technologies are combined with lotion, cream, or liquid compositions, which in turn may be or comprise emulsion compositions. In some embodiments, provided technologies do not utilize skin disrupting technologies, such as chemical penetration enhancing agents.

In some embodiments, the present invention utilizes emulsion compositions comprising large agents that are particularly effective and/or useful in medical contexts, e.g., for therapeutic purposes. In some embodiments, particular emulsion compositions are particularly effective and/or useful for topical administration of agents to a subject in need thereof. In some embodiments emulsion compositions may comprise of one or more large agents.

In some embodiments, an emulsion may be formulated into a composition suitable for topical administration on the skin. In some embodiments, a composition suitable for topical administration may be a lotion, cream, powder, ointment, liniment, gel, or drops.

In some embodiments, emulsion formulations comprise water, medium chain triglyceride, span 65, polysorbate 80, methylparaben, and propylparaben. In some embodiments, macroemulsion formulations comprise water, medium chain triglyceride, span 65, and polysorbate 80.

### Formulations

In some embodiments, a large agent (e.g., a botulinum toxin) may be provided and/or utilized in accordance with the present disclosure in a composition formulated for appropriate administration (e.g., for topical administration such as; for example, topical administration to a skin surface, e.g., to achieve transdermal delivery, or, alternatively, in some embodiments, for parenteral administration).

In some embodiments, a large agent composition (e.g., a botulinum toxin composition) may be or comprise an emulsion composition, such as a nanoemulsion composition.

In some embodiments, a large agent composition (e.g., a botulinum toxin composition) may be formulated as a cream, drops, foam, gel, liniment, liquid, lotion, ointment, powder, spray, etc. (e.g., which may, in some embodiments, be or comprise an emulsion composition such as a nanoemulsion composition).

It will be appreciated by those of ordinary skill in the art that compositions for topical administration may be formulated, for example, as skin softener, nutrition lotion type emulsion, cleansing lotion, cleansing cream, skin milk, emollient lotion, massage cream, emollient cream, make-up base, facial pack or facial gel, cleaner formulation such as shampoos, rinses, body cleanser, hair-tonics, or soaps, or dermatological composition such as lotions, ointments, gels, creams, patches or sprays. In some embodiments, compositions for topical administration are not formulated for administration to mucous membranes (e.g., are inappropriate for application to mucous membranes and/or are not formulated to deliver an appropriate amount of large agent to or across mucous membranes).

In some embodiments, a formulation for use in accordance with the present disclosure (e.g., for topical administration) may comprise one or more of purified water, methylparaben, mineral oil, isopropyl myristate, white petrolatum, emulsifying wax, and propylparaben; in some embodiments a formulation for use in accordance with the present disclosure (e.g., for topical administration) may comprise one or more of purified water, mineral oil, isopropyl myristate, white petrolatum, and emulsifying wax.

In general, formulations for use in accordance with the present disclosure may be prepared by any appropriate method, for example as known or hereafter developed in the art of pharmacology. In general, such preparatory methods include a step of bringing an provided composition into association with one or more excipients, and then, if necessary and/or desirable, shaping and/or packaging into an appropriate form for administration, for example as or in a single- or multi-dose unit.

In some embodiments, useful may be prepared, packaged, and/or sold in bulk, as a single unit dose, and/or as a plurality of single unit doses. As used herein, a “unit dose” is a discrete amount of a pharmaceutical composition comprising a predetermined amount of the provided composition. In some embodiments, a formulation comprises and/or delivers a single dose of a relevant active agent (e.g., a large agent such as botulinum toxin), or a convenient fraction thereof.

In some embodiments, appropriate excipients for use in compositions (e.g., pharmaceutically and/or cosmetically acceptable compositions) may, for example, include one or more excipients such as solvents, dispersion media, granulating media, diluents, or other liquid vehicles, dispersion or suspension aids, surface active agents and/or emulsifiers, isotonic agents, thickening or emulsifying agents, preservatives, solid binders, lubricants, disintegrating agents, binding agents, preservatives, buffering agents and the like, as suited to the particular dosage form desired. In some embodiments, excipients such as cocoa butter and/or suppository waxes, coloring agents, coating agents, sweetening, flavoring, and/or perfuming agents can be utilized. Remington's The Science and Practice of Pharmacy, 21^{st} Edition, A. R. Gennaro (Lippincott, Williams & Wilkins, Baltimore, Md., 2005; incorporated herein by reference) discloses various excipients used in formulating pharmaceutical compositions and known techniques for the preparation thereof.

In some embodiments, an appropriate excipient (e.g., a pharmaceutically and/or cosmetically acceptable excipient) is at least 95%, at least 96%, at least 97%, at least 98%, at least 99%, or 100% pure, in some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or other international Pharmacopoeia.

Particular exemplary formulations may be prepared, for example, as cosmetic formulation products such as skin softeners, nutritional lotion type emulsions, cleansing lotions, cleansing creams, skin milks, emollient lotions, massage creams, emollient creams, make-up bases, facial packs or facial gels, cleaner formulations such as shampoos, rinses, body cleansers, hair-tonics, or soaps, or dermatological compositions such as lotions, ointments, gels, creams, liniments, patches, deodorants, or sprays. In some embodiments, compositions for topical administration are not formulated for administration to mucous membranes (e.g., are inappropriate for application to mucous membranes and/or are not formulated to deliver an appropriate amount of large agent to or across mucous membranes).

In some embodiments, the present invention provides particular cream and/or lotion formulations as described herein. In some embodiments, provided cream and/or lotion formulations comprise water. In some embodiments, provided cream and/or lotion formulations comprise methylparaben. In some embodiments, provided cream and/or lotion formulations comprise mineral oil. In some embodiments, provided cream and/or lotion formulations comprise isopropyl myristate. In some embodiments, provided cream and/or lotion formulations comprise white petrolatum. In some embodiments, provided cream and/or lotion formulations comprise emulsifying wax. In some embodiments, provided cream and/or lotion formulations comprise propylparaben. In some embodiments, provided cream and/or lotion formulations do not comprise any parabens. In some embodiments, provided cream and/or lotion formulations do not comprise methylparaben. In some embodiments, provided cream and/or lotion formulations do not comprise propylparaben.

An exemplary lotion formulation is provided in Table 1.

TABLE 1
Exemplary Cream and/or Lotion Formulation

| % w/w | Ingredient |
|---|---|
| 72.00 | Purified Water |
| 0.200 | Methylparaben |
| 5.00 | Mineral Oil |
| 5.00 | Isopropyl Myristate |
| 2.000 | White Petrolatum |
| 15.00 | Emulsifying Wax |
| 0.800 | Propylparaben |
| 100 | TOTAL |

In some embodiments, cream and/or lotion formulations may be useful for topical and/or transdermal administration. The present disclosure encompasses the recognition that provided cream and/or lotion formulations can be particularly useful for delivery of agents to the dermal layer of the skin. In some embodiments, provided cream and/or lotion formulations are formulated for topical and/or transdermal delivery to a subject in need thereof. In some embodiments, provided cream and/or lotion formulations are administered to a subject in need thereof via topical and/or transdermal delivery.

In some embodiments, provided compositions are formulated with cosmetically acceptable components. For example, in some embodiments, provided compositions are formulated with water and also any cosmetically acceptable solvent, in particular, monoalcohols, such as alkanols having 1 to 8 carbon atoms (like ethanol, isopropanol, benzyl alcohol and phenylethyl alcohol), polyalcohols, such as alkylene glycols (like glycerine, ethylene glycol and propylene glycol), and glycol ethers, such as mono-, di-, and tri-ethylene glycol monoalkyl ethers, for example, ethylene glycol monomethyl ether and diethylene glycol monomethyl ether, used singly or in a mixture. Such components can be present, for example, in proportions of up to as much as 60%, 70%, 80%, or 90% by weight, relative to the weight of the total composition.

In some embodiments, provided compositions for topical administration include one or more cosmetically acceptable components that impart appearance attributes desirable or appropriate for a subject to which the composition is to be applied (e.g, a matte appearance, which may be particularly desirable or appropriate for administration to subjects having greasy skin).

In some embodiments, provided compositions are formulated with at least one cosmetically acceptable filler material, for example, in order to obtain a matte product, which may be especially desired for individuals with greasy skin.

In some embodiments, a botulinum toxin composition formulated for administration (e.g., as a cream or lotion) comprises between about 1 to about 200,000 Units botulinum toxin per mL, or between about 1 to about 100,000 Units botulinum toxin per mL, or between about 1 to about 50,000 Units botulinum toxin per mL, or between about 500 to about 20,000 Units botulinum toxin per mL, or between about 100 to about 2,000 Units botulinum toxin per mL, or between about 50 to about 500 Units botulinum toxin per mL, or between about 25 to about 400 Units botulinum toxin per mL. In some embodiments, a botulinum toxin composition formulated for administration (e.g., as a cream or lotion) comprises between about 2 to about 40,000 Units botulinum toxin per mL, or about 12,000 Units botulinum toxin per mL, or between about 100 to about 2,000 Units botulinum toxin per mL, or between about 50 to about 1,000 Units botulinum toxin per mL.

Those of ordinary skill in the art will appreciate that units herein relate to Units that are biologically equivalent or bioactively equivalent to Units defined by commercial manufacturers of botulinum toxin.

In some embodiments, the present invention provides a topical formulation of botulinum toxin that avoids potential complications including, but not limited to, systemic toxicity or botulism poisoning. In some embodiments, dosages of botulinum toxin (including types A, B, C, D, E, F, or G or botulinum that is genetically engineered or chemically modified to act longer or shorter in duration than botulinum toxin serotype A) can range from as low as about 1 unit to as high as about 50,000 units, with minimal risk of adverse side effects. The particular dosages may vary depending on the condition being treated and therapeutic regime being utilized. For example, treatment of subdermal, hyperactive muscles may require high transdermal dosages (e.g., 1000 units to 20,000 units) of botulinum toxin. In comparison, treatment of neurogenic inflammation or hyperactive sweat glands may require relatively small transdermal dosages (e.g. about 1 unit to about 1,000 units) of botulinum toxin.

In some embodiments, a large agent composition may be formulated and delivered in combination with MSC as described herein so that systemic delivery is achieved; in some embodiments, provided compositions may be formulated and/or delivered so that local, but not systemic, delivery is achieved.

In some embodiments, large agent compositions may be formulated and delivered in combination with MSC as described herein so that systemic delivery is achieved; in some embodiments, provided compositions may be formulated and/or delivered so that local, but not systemic, delivery is achieved.

In some embodiments, compositions suitable for topical formulation comprise a penetration enhancing agent. In some embodiments, a penetration enhancing agent degrades, disrupts and/or damages skin structure(s) and/or skin. In some embodiments, a penetration enhancing agent does not degrade, disrupt and/or damage skin structure(s) and/or skin. In some embodiments, a penetration enhancing agent is an irritant. In some embodiments, a penetration enhancing agent is not an irritant.

The present disclosure specifically demonstrates effective and efficient delivery of a large agent (and, in particular, a large biologic agent, such as botulinum toxin and/or antibody agent and/or prophylactic agent such as a vaccine) to the dermis using provided compositions in combination with MSC as described herein. For example, in some embodiments, the present invention provides methods comprising administration of a composition as described herein without clinically significant side effects. To give but one example, when topical delivery is contemplated, clinically significant side effects include, but are not limited to, unwanted systemic side effects, damage to nervous tissue underlying the dermis (e.g., neuronal paralysis), unwanted effects on muscles (e.g., muscle paralysis), and/or undesirable blood levels of large agent, etc.

Those of ordinary skill in the art reading the present disclosure will appreciate that, in some embodiments, provided compositions may be incorporated into a device such as, for example, a patch. A variety of transdermal patch structures are known in the art; those of ordinary skill will appreciate that provided compositions may readily be incorporated into any of a variety of such structures. In some embodiments, a transdermal patch may comprise a plurality of needles extending from one side of the patch that is applied to the skin, wherein needles extend from the patch to project through the stratum corneum of the skin. In some embodiments, needles do not rupture a blood vessel. In some embodiments, needles do not penetrate deeply enough to reach nerves in the dermis of the skin.

In some embodiments, a transdermal patch includes an adhesive. Some examples of adhesive patches are well known (for example, see U.S. Design Patent 296,006; and U.S. Pat. Nos. 6,010,715; 5,591,767; 5,008,110; 5,683,712; 5,948,433; and 5,965,154; all of which are incorporated herein by reference). Adhesive patches are generally characterized as having an adhesive layer, which will be applied to a patient's skin, a depot or reservoir for holding a provided composition, and an exterior surface that prevents leakage of the provided composition from the depot. The exterior surface of a patch may be non-adhesive.

In accordance with some embodiments, a large agent composition may be a patch; in some embodiments, an incorporated large agent remains stable for extended periods of time. In some embodiments, a provided composition may be incorporated into a polymeric matrix that stabilizes an large agent, and permits the agent to diffuse from the matrix and the patch. In some embodiments, a large agent composition may be incorporated into an adhesive layer of a patch so that once the patch is applied to the skin, the provided composition may diffuse through the skin. In some embodiments, an adhesive layer may be heat-activated where temperatures of about 37 CC cause the adhesive to slowly liquefy so that the agent diffuses through the skin. The adhesive may remain tacky when stored at less than 37° C., and once applied to the skin, the adhesive loses its tackiness as it liquefies.

In some embodiments, a large agent composition can be provided in a depot in a patch so that pressure applied to the patch causes the provided composition to be directed out of the patch through microneedles and through the stratum corneum. Exemplary embodiments of microneedles are described above. Suitable devices for use in administering provided compositions intradermally include devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof.

In some embodiments, for example in order to prolong the effect of a large agent composition, it may be desirable to slow absorption of a provided composition into the skin. In some embodiments, this may be accomplished by use of a liquid suspension of crystalline or amorphous material with poor water solubility. The rate of absorption of a provided composition then depends upon its rate of dissolution which, in turn, may depend upon crystal size and crystalline form. In some embodiments, depending upon the ratio of provided composition to polymer and the nature of the particular polymer employed, the rate of provided composition release can be controlled. Examples of other biodegradable polymers include poly(orthoesters) and poly(anhydrides).

In some embodiments, provided formulations comprise a mixture of a provided emulsion composition (e.g., nanoemulsion composition) and one or more pharmaceutically acceptable excipients. In some embodiments; cream and/or lotion formulations comprise a mixture of a provided nanoemulsion composition and/or a saline solution.

In some embodiments, provided compositions comprise provided nanoemulsion compositions. In some embodiments, provided compositions are cream and/or lotion formulations. In some embodiments, provided cream and/or lotion formulations comprise nanoemulsion compositions. In some embodiments, compositions comprise provided nanoemulsion compositions but are not cream and/or lotion formulations. In some embodiments, suitable compositions are formulated into creams and/or lotions but do not comprise a nanoemulsion composition.

In some embodiments, provided compositions comprise a mixture of a provided nanoemulsion composition and one or more pharmaceutically acceptable excipients, e.g, for topical and/or transdermal (e.g., by lotions; creams, powders, ointments, liniments, gels, drops, etc.) administration.

In some embodiments, for nanoemulsion compositions comprising a known therapeutic agent and/or independently active biologically active agent, such nanoemulsion compositions are arranged and constructed and administered in combination with MSC such that an amount of therapeutic agent is delivered to a desired target site (e.g., to epidermal and/or dermal structures) that is sufficient to treat a condition or disorder. In some embodiments, provided nanoemulsion compositions are arranged and constructed (e.g., through selection and/or combination of agents, structure of composition, etc.) such that they achieve a desired therapeutic effect upon administration to the skin. In some embodiments, provided nanoemulsion compositions are arranged and constructed such that they do not induce unwanted clinical effects inside and/or outside of a desired site of action (e.g., surface of skin, dermis, etc.). In some embodiments, provided nanoemulsion compositions are arranged and constructed and administered in combination with MSC such that they have systemic effects.

In some embodiments, provided compositions may be formulated and delivered in combination with MSC so that systemic delivery is achieved; in some embodiments, provided compositions may be formulated and/or delivered so that local, but not systemic, delivery is achieved.

The present disclosure specifically demonstrates effective and efficient delivery of a therapeutic agent (and, in particular, a large biologic agent, such as botulinum toxin or antibody agent) to the dermis using provided compositions in combination with MSC. For example, in some embodiments, the present invention provides methods comprising administration of a composition as described herein without clinically significant side effects. To give but one example, when topical delivery is contemplated, clinically significant side effects include, but are not limited to, unwanted systemic side effects, damage to nervous tissue underlying the dermis (e.g., neuronal paralysis), unwanted effects on muscles e.g., muscle paralysis), and/or undesirable blood levels of therapeutic agent, etc.

In some embodiments, the present invention provides topical formulations of a large agent (e.g., botulinum toxin or antibody agent, etc) that allow the agent to permeate through a subject's skin without permeating in significant amount through a blood vessel. For example, in some embodiments of the invention, less than about 25%, less than about 20%, less than about 15%, less than about 10%, less than about 5%, less than about 4%, less than about 3%, less than about 2%, or less than about 1% of the agent present in the formulation permeates into a blood vessel upon application in accordance with the present disclosure.

Those of ordinary skill in the art will appreciate that inventive compositions that achieve transdermal administration of botulinum toxin or antibody agents may be incorporated into a device such as, for example, a patch, a roller, a pen, a stamp, and so forth.

### Uses

In some embodiments, the present disclosure provides large agent therapies (e.g., botulinum toxin therapies) that involve administration of one or more doses of a relevant large agent composition (e.g., a botulinum toxin composition) combination with microneedling (e.g., with microneedle skin conditioning) in accordance with a regimen that has been demonstrated to achieve a delayed onset of effect and/or a delayed peak effect as described herein (e.g., a detectable onset that is later than about 1, about 2, about 3, about 4, about 5, about 6, about 7, about 8, about 9, about 10, about 11, about 12, about 13, about 14 or more days and/or a peak effect later than about 2, about 3, about 4, about 5, about 6, about 7, about 8, about 9, about 10, about 11 months, or even later than a year of more, after administration) for one or more doses within the regimen.

The present disclosure provides an insight that such technologies are particularly useful in certain contexts (e.g., for the treatment of certain subjects and/or sites thereon—in particular those subjects suffering from particular diseases disorders or conditions and/or site(s) reflective thereof, for which delayed peak effect and/or extended duration of response may be particularly desirable.

The present disclosure demonstrates that certain dosing regimens (e.g., with extended periods of time between doses—in light of the extended duration of effect documented herein) can surprisingly be effective, and even particularly useful in certain contexts (e.g., when administered to subjects and/or sites suffering from a disease, disorder or condition with respect to which delayed peak effect and/or extended duration of response may be particularly desirable, including for reasons described herein).

The present invention provides, among other things, technologies for administering large agents, e.g., botulinum toxin or antibody agents, improving delivery transdermally, and/or improving bioavailability of such large agents, by incorporating one or more large agents into one or more emulsion compositions which are then administered in combination with MSC as described herein, which technologies surprisingly provide delayed peak effect and/or extended duration of action relative to certain alternative technologies for delivering the relevant large agent. The present inventors have surprisingly found that not only are transdermal permeation and bioavailability of botulinum toxin or antibody agents incorporated into nanoemulsion compositions is dramatically improved when used in combination with MSC using microneedles or microneedle array with relatively low microneedle density, or with relatively small microneedle puncture size (e.g., puncture size per microneedle, cross-sectional area of each microneedle), but also a benefit of the instant invention is the ability to administer such large agents intradermally while minimizing irritation or damage to the skin, and, moreover, certain unexpected results can be achieved, so that the present disclosure teaches that topical application of emulsion compositions that comprise and/or deliver a large agent, in combination with MSC, in particularly useful in certain contexts (e.g., when administered to particular subjects and/or sites). Still further, the present disclosure establishes that certain dosing regimens (e.g., in which individual administrations of a formulation—e.g., comprising an emulsion composition such as a nanoemulsion—that comprises and/or delivers a large agent are separated by extended periods of time, e.g., in light of the extended duration of effect).

In some embodiments, a large agent is botulinum toxin. In some embodiments, a botulinum toxin emulsion composition (e.g., in a formulation as described herein) is applied directly to the skin and for absorption through the epidermal layers before MSC. In some embodiments, such a botulinum toxin emulsion composition is applied directly to the skin and for absorption through the epidermal layers after MSC. In some embodiments, a botulinum toxin emulsion composition is applied directly to the skin and for absorption through the epidermal layers at substantially the same time as MSC.

In some embodiments, a botulinum toxin emulsion (e.g., nanoemulsion) composition in combination with MSC can penetrate the top layer of the skin, including the stratum corneum, dermal pores, and/or dermal glands, without the use of a penetration enhancing agent. In some embodiments, a botulinum emulsion composition in combination with MSC can penetrate the top layer of the skin, including the stratum corneum, dermal pores, and/or dermal glands, without the use of degradant, irritant, and/or abrasive agents.

In some embodiments, an antibody agent emulsion composition in combination with MSC can penetrate the top layer of the skin, including the stratum corneum, dermal pores, and/or dermal glands, without the use of a penetration enhancing agent. In some embodiments, a large agent is an antibody agent. In some embodiments, an antibody agent emulsion composition is applied directly to the skin and for absorption through the epidermal layers before MSC. In some embodiments, an antibody agent emulsion composition is applied directly to the skin and for absorption through the epidermal layers after MSC. In some embodiments, an antibody agent emulsion composition is applied directly to the skin and for absorption through the epidermal layers at substantially the same time as MSC. In some embodiments, an antibody agent emulsion composition is applied directly to the skin and for absorption systemically.

In some embodiments, an antibody agent emulsion composition in combination with MSC can penetrate the top layer of the skin, including the stratum corneum, dermal pores, and/or dermal glands, without the use of degradant, irritant and/or abrasive agents.

Diseases. Disorders and Conditions

Technologies provided by the present disclosure are useful in the treatment and/or prevention of any of a variety of diseases, disorders, and/or conditions, particularly including certain systemic or dermatologic diseases, disorders or conditions.

In some embodiments, the present invention provides technologies for treating and/or preventing diseases, disorders, or conditions associated with activity of sweat and/or sebaceous glands. In some embodiments, the present invention provides technologies for treating and/or preventing diseases, disorders, or conditions associated with infection. In some embodiments, the present invention provides technologies for treating and/or preventing diseases, disorders, or conditions associated with inflammation. In some embodiments, the present invention provides technologies for treating and/or preventing diseases, disorders, or conditions associated with cancer. In some embodiments, the present invention provides technologies for treating and/or preventing diseases, disorders, or conditions which are systemic. In some embodiments, the present invention provides technologies for treating and/or preventing diseases, disorders, or conditions which are autoimmune. In some embodiments, the present invention provides technologies for treating and/or preventing diseases, disorders or conditions associated with the epidermal and/or dermal level of the skin. In some embodiments, the present invention provides technologies for treating and/or preventing diseases, disorders or conditions of the eye.

In some embodiments, the present invention provides technologies for treating and/or preventing one or more of acne, unwanted sweating, body odor, hyperhidrosis, bromhidrosis, chromhidrosis, rosacea, hair loss, psoriasis, actinic keratosis, eczematous dermatitis (e.g., atopic dermatitis, etc.), excess sebum-producing disorders e.g., seborrhea, seborrheic dermatitis, etc.), burns, Raynaud's phenomenon, lupus erythematosus, hyperpigmentation disorders (e.g., melasma, etc.), hypopigmentation disorders vitiligo, etc.), skin cancer (e.g., squamous cell skin carcinoma, basal cell skin carcinoma, etc.), dermal infection (e.g., bacterial infection, viral infection, fungal infection, etc.), facial wrinkles, (e.g., wrinkles involving the forehead, glabellar, rhytids and/or periorbital regions), headache, unsightly facial expressions (e.g., due to overactivity of underlying facial musculature), neck lines, hyperfunctional facial lines, hyperkinetic facial lines, platysma bands, décolletage wrinkles, neuromuscular disorders and conditions involving muscular spasm and/or contracture (including various forms of facial palsy, cerebral palsy, blepharospasm, facial contracture), dystonia, prostate hyperplasia, headache, strabismus, hemifacial spasm, tremor, spasticity such as that resulting from multiple sclerosis, retroorbital muscle, various ophthalmologic and urologic conditions (e.g., penile and/or bladder disorders, and penile and scrotal wrinkles), and/or combinations thereof.

In certain embodiments, provided technologies may be particularly useful in the treatment of wrinkles, including, for example wrinkles involving the forehead, glabellar, rhytids and/or periorbital regions including Crow's Feet wrinkles), unsightly facial expressions (e.g., due to overactivity of underlying facial musculature), neck lines, hyperfunctional facial lines, hyperkinetic facial lines, platysma bands, déolletage wrinkles, hand wrinkles, foot wrinkles, breast wrinkles, penile wrinkles, and scrotal wrinkles.

In some embodiments, the present invention provides technologies for treating and/or preventing rheumatoid arthritis. In some embodiments, the present invention provides technologies for treating and/or preventing psoriatic arthritis. In some embodiments, the present invention provides technologies for treating and/or preventing osteoarthritis.

In some embodiments, the present invention provides technologies for treating and/or preventing lupus erythematosus. In some embodiments, the lupus erythematosus is systemic, discoid, drug-induced, or neonatal. In some embodiments, the present invention provides technologies for treating and/or preventing Crohn's disease. In some embodiments, the present invention provides technologies for treating and/or preventing inflammatory bowel disease. In some embodiments, the present invention provides technologies for treating and/or preventing ulcerative colitis.

In some embodiments, the present invention provides technologies for treating and/or preventing pulmonary disorders. In some embodiments, the pulmonary disorder may be asthma or chronic obstructive pulmonary disorder.

In some embodiments, the present invention provides technologies for treating and/or preventing amyloidosis. In some embodiments the amyloidosis is systemic or cutaneous.

In some embodiments, the present invention provides technologies for treating and/or preventing cancer. In some embodiments the cancer is of the skin, blood, breast, colon, or lung.

In some embodiments, the present invention provides technologies for treating and/or preventing dyslipidemia. In some embodiments the dyslipidemia is hypercholesterolemia.

In some embodiments, the present invention provides technologies for treating and/or preventing infection. In some embodiments, the infection is or is caused by C. difficile or Staphylococcus.

In some embodiments, the present invention provides technologies for treating and/or preventing pain. In some embodiments the pain is associated with arthritis. In some embodiments the arthritis is rheumatoid arthritis, psoriatic arthritis, or osteoarthritis.

In some embodiments, the present invention provides technologies for treating and/or preventing neurologic conditions. In some embodiments the neurological condition is Alzheimer's Disease, Parkinson's Disease, or stroke.

In certain embodiments, provided technologies are useful in the treatment and/or prevention of one or more diseases, disorders, and conditions such as, for example, certain dermatologic conditions (e.g., acne, rosacea), certain eye disorders e.g., blepharospasm, strabismum, etc), various muscular and/or movement disorders (e.g., cervical dystonia, muscle contracture, muscle spasms, muscle stiffness, torticollis etc), certain bladder and/or bowel disorders (e.g., leaking urine, overactive bladder, including in subjects who cannot tolerate side effects associated with other therapies; urgency of urination; etc), migraines, sweat disorders (e.g., bromhidrosis, chromhidrosis, hyperhidrosis, etc), wrinkles, etc.

In some embodiments, the present disclosure provides technologies that involve administration according to a dosing regimen sufficient to achieve a reduction in the degree and/or prevalence of a relevant dermatologic condition of at least about 20%; in some embodiments according to a dosing regimen sufficient to achieve a of at least about 25%; in some embodiments according to a dosing regimen sufficient to achieve a reduction of at least about 30%; in some embodiments according to a dosing regimen sufficient to achieve a reduction of at least about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%; about 75%, about 76%, about 77%, about 78%, about 79%, about 80%, about 81%, about 82%, about 83%, about 84%, about 85%, about 86%, about 87%, about 88%, about 89%, about 90%, or more.

In some embodiments, the present invention involves administration of at least one provided composition, administered in combination with MSC, according to a dosing regimen sufficient to achieve a reduction in the degree and/or prevalence of a relevant dermatologic condition of at least about 20% in a specified percentage of a population of patients to which the composition was administered; in some embodiments according to a dosing regimen sufficient to achieve a of at least about 25% in a specified percentage of a population of patients to which the composition was administered; in some embodiments according to a dosing regimen sufficient to achieve a reduction of at least about 30% in a specified percentage of a population of patients to which the composition was administered; in some embodiments according to a dosing regimen sufficient to achieve a reduction of at least about 31%, about 32%, about 33%, about 34%, about 35%, about 36%, about 37%, about 38%, about 39%, about 40%, about 41%, about 42%, about 43%, about 44%; about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%; about 67%, about 68%, about 69%, about 70%; about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90% or more in a specified percentage of a population of patients to which the composition was administered. In some embodiments, the specified percentage of population of patients to which the composition was administered is at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. To give but a few illustrative examples, in some embodiments, the present invention involves administration of at least one provided composition according to a dosing regimen sufficient to achieve a reduction in the degree and/or prevalence of a relevant dermatologic condition of at least about 20% in at least about 50% of the population of patients to which the composition was administered. In some embodiments, the present invention involves administration of at least one provided composition according to a dosing regimen sufficient to achieve a reduction in the degree and/or prevalence of a relevant dermatologic condition of at least about 30% in at least about 50% of the population of patients to which the composition was administered.

The present invention provides technologies for treating and/or preventing a dermatologic condition comprising administration of a provided composition in combination with MSC to a subject suffering from, susceptible to, and/or displaying symptoms of the dermatologic condition. In some embodiments, provided compositions for treatment of a dermatologic condition as described herein are formulated for any route of administration described herein. In some embodiments, provided compositions are formulated for topical administration. In some embodiments, provided compositions are formulated into a cream, liniment, lotion, gel, shampoo, conditioner, sunscreen, deodorant, and/or antiperspirant (e.g., as a roll-on, solid stick, gel, cream, aerosol, etc.), etc, as appropriate to the condition being treated.

In some embodiments, such a provided composition is administered locally in combination with MSC to an affected site (e.g., axillae, hands, feet, scalp, hair follicle, face, neck, back, arms, chest, legs, groin, crotch, etc., as appropriate to a particular condition being treated), in some embodiments, local administration is achieved by topical administration in combination with MSC.

Combinations

In some embodiments, technologies described herein may be utilized to administer a large agent (e.g., a botulinum toxin) in combination with another agent and/or with another treatment.

In some embodiments, provided technologies may be utilized to administer a plurality of large agents (e.g., a botulinum toxin in combination with one or more other large agents such as one or more antibodies).

In some embodiments, provided technologies may be utilized in combination with one or more penetration enhancing technologies (e.g., one or more penetration enhancing agents); in some embodiments, no penetration enhancing agents are utilized. In some embodiments, provided technologies may be utilized with one or more penetration enhancing agents that are not irritants and/or do not degrade, disrupt and/or damage skin structure(s) and/or skin. In some embodiments, a non-irritating penetration enhancing agent may be selected from, for example, co-peptides, carrier molecules, and carrier peptides. In some embodiments a carrier molecule is positively charged. In some embodiments, a carrier molecule may be a co-peptide. In some embodiments, a carrier molecule may be a long-chain positively charged polypeptide or a positively charged nonpeptidyl polymer, for example, a polyalkyleneimine. In some embodiments a carrier peptide may be a cationic peptide. In some embodiments, a carrier peptide is a positively charged carrier with the sequence RKKRRQRRRG-(K)₁₅-GRKKRRQRRR. In some embodiments, a carrier molecule may be one disclosed in U.S. Patent Publication 2010/0168023 or U.S. Patent Publication 2009/0247464 the contents of which are herein incorporated by reference in their entireties.

In some embodiments, provided technologies may be utilized in combination with one or more therapies that acts on or in skin and/or that imparts a therapeutic and/or cosmetic effect. In some embodiments, biologically active agents utilized in combination with an antibody agent as described herein may be an agent that acts on or in skin and/or that imparts a therapeutic and/or cosmetic effect. For example, provided technologies may be utilized in combination with one or more anesthetics (e.g, lidocaine), steroids (e.g., hydrocortisone), and/or retinoids (e.g., retin A), cosmetic agents such as dermal fillers (such as hyaluronic acid or other elastic materials), collagen, and/or silicone.

### Subjects

Technologies of the present disclosure are suitable for both human and veterinary use. Subjects who can benefit from treatment with technologies describe herein particularly include subjects suffering from a disease, disorder, or condition as described herein. In particular embodiments, subjects are suffering from a diseases, disorder or condition that is cosmetic and/or includes a visually-apparent feature, effect, or characteristic. Among other contributions of the present disclosure is an insight that, for certain such subjects, delayed peak effect and/or extended duration of effect may be particularly desirable.

In some embodiments, an administration site is the skin overlying a muscle or muscle group of a subject. In some embodiments, the site is hairless. In some embodiments, the site is on the torso. In some embodiment the site is on the back. In some embodiments the site is on the chest. In some embodiments, the site is on the buttocks. In some embodiments, the site is on the crotch. In some embodiments, the site is on the groin. In some embodiments, the site is on the head. In some embodiments the site is on the scalp. In some embodiments, the site is on the face. In some embodiments the site is on the neck. In some embodiments the site is on the décolleté. In some embodiments, the site is in the armpit. In some embodiments, the site is on the axillae. In some embodiments the site is on the hands. In some embodiments the site is on the feet. In some embodiments the site is on the arms. In some embodiments the site is on the legs. In some embodiments, the site is not a mucous membrane.

In some embodiments the site is affected by a dermatologic condition. In some embodiments the site is the skin overlying a muscle or muscle group affected by a neuromuscular condition. In some embodiments, the length of the microneedles used in MSC is adjusted based on skin thickness of the treatment site.

In many embodiments, a subject is suffering from wrinkles and/or technologies as described herein are applied to a wrinkle site (e.g, to a site of wrinkled skin).

In some embodiments, provided technologies can achieve controlled and/or improved delivery of active agents efficiently and specifically to biologically relevant target sites (e.g., particular tissues, locations within the skin, cells, etc.). In some embodiments, the present invention demonstrates controlled delivery and/or achievement of therapeutic effect in a certain biologically relevant target site without significant side effects associated with delivery to other areas.

In some embodiments, provided technologies can improve delivery and/or bioavailability of active agents efficiently and delivery specifically to the dermis, and/or have cosmetic and/or therapeutic effects upon administration to the skin of a subject. In some embodiments, the present invention demonstrates improved delivery and/or bioavailability through dermal delivery and/or achievement of therapeutic effect without significant side effects associated with delivery to other areas (e.g., to subdermal or extradermal structures and/or to tissues other than dermis). In some embodiments, provided technologies can improve transdermal delivery and/or bioavailability of active agents, such as therapeutic agents (e.g., botulinum toxins, antibody agents, etc.).

In some embodiments, a large agent administered in accordance with the present disclosure penetrates the skin within about 1, 2, 3, 4, 5, 6, 7, 8, 9, or 10 minutes of administration. In some embodiments, a large agent penetrates the skin within about 5 to about 60 minutes of administration. In some embodiments, a large agent penetrates the skin within about 5 to about 12 minutes of administration. In some embodiments, a large agent penetrates the skin within about 5 to about 15 minutes of administration. In some embodiments, a large agent penetrates the skin within about 15 to about 30 minutes of administration. In some embodiments, a large agent penetrates the skin within about 1 hour of administration. In some embodiments, a large agent penetrates the skin within about 2 hours of administration. In some embodiments, a large agent penetrates the skin within about 3 hours of administration. In some embodiments, a large agent penetrates the skin within about 4 hours of administration. In some embodiments, a large agent penetrates the skin within about 5 hours of administration. In some embodiments, a large agent penetrates the skin within about 6 hours of administration.

In some embodiments, a large agent penetrates a layer of the skin within about 1, 2, 3, 4, 5, 6, 7, 8, 9, or 10 minutes of administration. In some embodiments, a large agent penetrates a layer of the skin within about 5 to about 60 minutes of administration. In some embodiments, a large agent penetrates a layer of the skin within about 5 to about 12 minutes of administration. In some embodiments, a large agent penetrates a layer of the skin within about 5 to about 15 minutes of administration. In some embodiments, a large agent penetrates a layer of the skin within about 15 to about 30 minutes of administration. In some embodiments, a large agent penetrates a layer of the skin within about 1 hour of administration. In some embodiments, a large agent penetrates a layer of the skin within about 2 hours of administration. In some embodiments, a large agent penetrates a layer of the skin within about 3 hours of administration. In some embodiments, a large agent penetrates a layer of the skin within about 4 hours of administration. In some embodiments, a large agent penetrates a layer of the skin within about 5 hours of administration. In some embodiments, a large agent penetrates a layer of the skin within about 6 hours of administration.

In some embodiments, a large agent penetrates the top layer of the skin within about 1, 2, 3, 4, 5, 6, 7, 8, 9, or 10 minutes of administration. In some embodiments, a large agent penetrates the top layer of the skin within about 5 to about 60 minutes of administration. In some embodiments, a large agent penetrates the top layer of the skin within about 5 to about 12 minutes of administration. In some embodiments, a large agent penetrates the top layer of the skin within about 5 to about 15 minutes of administration. In some embodiments, a large agent penetrates the top layer of the skin within about 15 to about 30 minutes of administration. In some embodiments, a large agent penetrates the top layer of the skin within about 1 hour of administration. In some embodiments, a large agent penetrates the top layer of the skin within about 2 hours of administration. In some embodiments, a large agent penetrates the top layer of the skin within about 3 hours of administration. In some embodiments, a large agent penetrates the top layer of the skin within about 4 hours of administration. In some embodiments, a large agent penetrates the top layer of the skin within about 5 hours of administration. In some embodiments, a large agent penetrates the top layer of the skin within about 6 hours of administration.

In some embodiments, a large agent penetrates the top layer of the skin, including the stratum corneum, dermal pores, and/or dermal glands within about 1, 2, 3, 4, 5, 6, 7, 8, 9, or 10 minutes of administration. In some embodiments, a large agent penetrates the top layer of the skin, including the stratum corneum, dermal pores, and/or dermal glands within about 5 to about 60 minutes of administration. In some embodiments, a large agent penetrates the top layer of the skin, including the stratum corneum, dermal pores, and/or dermal glands within about 5 to about 12 minutes of administration. In some embodiments, a large agent penetrates the top layer of the skin, including the stratum corneum, dermal pores, and/or dermal glands within about 5 to about 15 minutes of administration. In some embodiments, a large agent penetrates the top layer of the skin, including the stratum corneum, dermal pores, and/or dermal glands within about 15 to about 30 minutes of administration. In some embodiments, a large agent penetrates the top layer of the skin, including the stratum comeum, dermal pores, and/or dermal glands within about 1 hour of administration. In some embodiments, a large agent penetrates the top layer of the skin, including the stratum corneum, dermal pores, and/or dermal glands within about 2 hours of administration. In some embodiments, a large agent penetrates the top layer of the skin, including the stratum corneum, dermal pores, and/or dermal glands within about 3 hours of administration. In some embodiments, a large agent penetrates the top layer of the skin, including the stratum corneum, dermal pores, and/or dermal glands within about 4 hours of administration. In some embodiments, a large agent penetrates the top layer of the skin, including the stratum corneum, dermal pores, and/or dermal glands within about 5 hours of administration. In some embodiments, a large agent penetrates the top layer of the skin, including the stratum corneum, dermal pores, and/or dermal glands within about 6 hours of administration.

### Kits

In some embodiments, the present invention provides pharmaceutical packs or kits including one or more emulsion compositions and one or more microneedle devices for use according to the present invention. In some embodiments, pharmaceutical packs or kits include preparations or pharmaceutical compositions containing provided compositions in one or more containers filled with optionally one or more additional ingredients of pharmaceutical compositions. In some embodiments, a pharmaceutical pack or kit includes an additional approved therapeutic agent (e.g., benzoyl peroxide for treatment of acne; aluminum compounds for treatment of hyperhidrosis; etc.) for use in combination therapies. In some embodiments, optionally associated with such container(s) can be a notice in the form prescribed by a governmental agency regulating the manufacture, use or sale of pharmaceutical products, which notice reflects approval by the agency of manufacture, use, or sale for human administration.

In some embodiments, kits are provided that include therapeutic reagents. As but one non-limiting example, provided compositions can be provided as topical formulations and administered as therapy in combination with use of a microneedling device. Pharmaceutical doses or instructions for self-administration therefor may be provided in a kit for administration to an individual suffering from or at risk for conditions or disorders, e.g., those associated with the dermal level of the skin.

In some embodiments, a kit may comprise (i) a provided composition; and (ii) at least one pharmaceutically acceptable excipient; and (iii) at least one device for microneedling the skin; and (iv) instructions for use. In some embodiments, the at least one device may comprise microneedles with relatively low microneedle density (e.g., in the range of about 2 microneedles/cm′ to about 50 microneedles/cm²). In some embodiments, for example, the at least one device may comprise microneedles with relatively small microneedle puncture size (e.g., puncture size per microneedle in the range of about 100 μm²/microneedle to about 30,000 pane/microneedle, puncture size per microneedle in the range of about 100 μm²/microneedle to about 60,000 μm²/microneedle).

### EXEMPLIFICATION

### Example 1: Effect of MSC Pre-Conditioning on Timing of Peak Therapeutic Effect and the Duration of Therapeutic Effect on Reduction of Crow's Feet Wrinkles

The present Example describes a study that assessed 1) timing of peak therapeutic effect and 2) duration of therapeutic effect on reduction of Crow's Feet wrinkles for botulinum toxin administered topically (in a nanoemulsion formulation) in combination with microneedle skin preconditioning.

The study included two test groups. Group A and Group B, of subjects who had moderate to severe Crow's Feet wrinkles. The Crow's Feet area of each subject was treated once topically with a botulinum toxin emulsion (e.g., a nanoemulsion) formulation (Group A) or its vehicle placebo control (Group B). Subjects in Groups A (N=9) and Group B (N=9) received skin pre-conditioning with identically matched sets of microneedle arrays. Administration of the topical formulation to the skin took about 5 minutes, by which time the topical formulation was fully absorbed into the skin. All subjects were pre-conditioned with eight microneedle impressions of a microneedle array prior to application of the botulinum toxin formulation.

The expected effect of a botulinum toxin treatment is to reduce Crow's Feet wrinkles at the site of the botulinum treatment. Wrinkle severity is measured using a five-point wrinkle scale (the Wrinkle Scale): 0=None, 1=Minimal, 2=Mild, 3=Moderate, 4=Severe by each of the investigators and subjects. As is commonly accepted in the field, a subject was considered to be a “responder” for purposes of peak effect when both the investigator and the subject assessed a reduction of two or more points in wrinkle severity on contraction, relative to baseline, and was considered to be a “responder” for purposes of duration of effect when the investigator assessed a reduction of one or more points in wrinkle severity on contraction, relative to baseline.

The study described in the present Example found that at Baseline, the average severity of the Crow's Feet wrinkles as measured by the Wrinkle Scale was approximately equal across Groups A and B.

In terms of peak effect, at a month after treatment, Group A had a responder rate of 11% and Group B had a responder rate of 0%; at two months after treatment, Group A had a responder rate of 11% and Group B had a responder rate of 0%; and at three months after treatment, Group A had a responder rate of 33% and Group B had a responder rate of 0%.

In terms of duration of therapeutic effect, at approximately six months after treatment, Group A had a responder rate of 56%. By comparison, literature reports of studies with a commercially available botulinum preparation administered by injection to Crow's Feet describe a responder rate of 15% at approximately six months using this same responder metric; other studies have found the median duration of therapeutic effect to treat wrinkles with commercially available botulinum preparations to be 3.5 to 4 months.

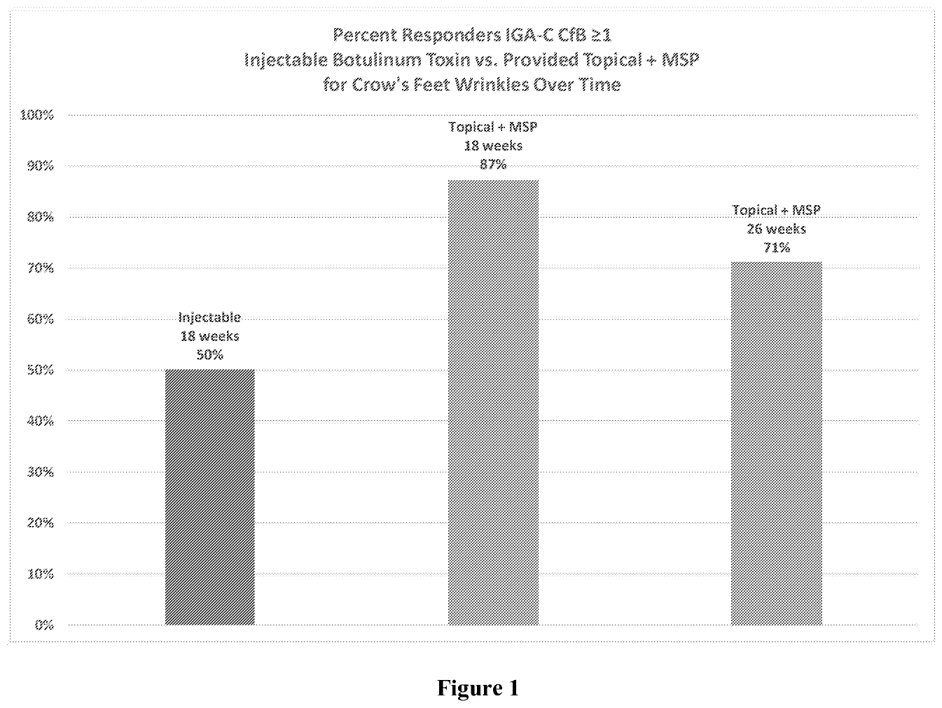

FIG. 1 presents a comparison of results achieved (specifically, responder rates observed) at particular time points (specifically at 18 weeks and 26 weeks) with Group A in the present study, as compared with a literature report of an approved botulinum toxin injectable product, administered according to its approved regimen for treatment of Crow's Feet. As can be seen, at 18 weeks, the injectable responder rate was below 50% whereas the responder rate for technologies described herein was above 80%. Furthermore, provided technologies achieved a responder rate that was higher (i.e., above 70%) at 26 weeks than was the injectable responder rate at 18 weeks.

The study described in this Example documents the unexpected result, described herein, that topical administration of botulinum toxin (e.g., in a nanoemulsion formulation), together with microneedle skin preconditioning, can increase the time at which peak therapeutic effect occurs, and furthermore can increase the duration of the therapeutic effect of a botulinum toxin therapy (e.g., for wrinkles).

### Example 2: Effect of MSC Pre-Conditioning on Timing of Peak Therapeutic Effect when Compared to Commercially Available Injectable Botulinum Toxins

The present Example describes a study that compared timing of peak therapeutic effect on reduction of Crow's Feet wrinkles for botulinum toxin administered topically (in a nanoemulsion formulation) in combination with microneedle skin preconditioning as described herein relative to reports that have described timing of peak therapeutic effect on reduction of Crow's Feet wrinkles for botulinum toxin administered by injection.

As described above, Group A (N=9) subjects who had moderate to severe Crow's Feet wrinkles were treated once topically with a botulinum toxin emulsion (in combination with skin pre-conditioning with microneedle arrays (specifically, with eight microneedle impressions of a microneedle array prior to application of the botulinum toxin formulation). Administration of the topical formulation to the skin took about 5 minutes, by which time the topical formulation was fully absorbed into the skin.

Results of this study were compared with literature reports describing treatment of subjects who had received approved injectable botulinum toxin therapy. Specifically, Group B (N=48) was treated with a first commercially available injectable botulinum toxin, and Group C (N=222) was treated with a different, second commercially available injectable botulinum toxin.

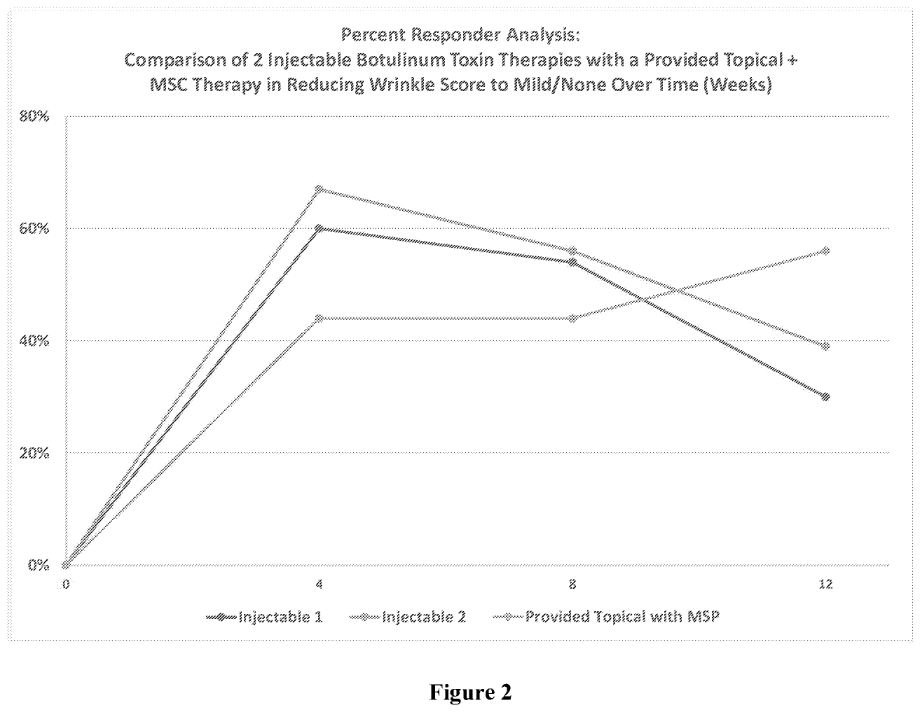

FIG. 2 compares the results of these studies. The expected effect of a botulinum toxin treatment is to reduce Crow's Feet wrinkles at the site of the botulinum treatment. For Group A, wrinkle severity was measured using a five-point wrinkle scale (the Wrinkle Scale): 0=None, 1=Minimal, 2=Mild, 3=Moderate, 4=Severe by each of the investigators. For Groups B and C, wrinkle severity had been measured using a four-point wrinkle scale (the Wrinkle Scale): 0=None, 1=Mild, 2=Moderate, 3=Severe by each of the investigators. A subject was considered to be a “responder” for purposes of peak effect when the investigator assessed the wrinkle score to be a Mild or more favorable score (e.g., None).

As can be seen with reference to FIG. 2, all three therapies ultimately achieved comparable peak effects; those skilled in the art would therefore appreciate that their relative regimens can be considered to be comparable notwithstanding that they may involve different absolute “doses” (whether considered in terms of units of botulinum toxin and/or amount [e.g., ng] of botulinum toxin) included in the administered single dose.

In terms of peak effect, FIG. 2 documents that, at one month after treatment, Group A had a responder rate of 44%, Group B had a responder rate of 60%, and Group C, 67%; at two months after treatment, Group A had a responder rate of 44%, Group B, 54%, and Group C, 56%; and at three months after treatment, Group A had a responder rate of 56%, Group B, 30%% and Group C, 39%. See FIG. 1. Peak effect was observed for Group A at a time point within a range of three months after administration of the dose of botulinum toxin; for Groups B and C, peak effect was observed at one month after administration.

The study described in this Example documents the unexpected result, described herein, that topical administration of botulinum toxin (e.g., in a nanoemulsion formulation), together with microneedle skin preconditioning, can increase the time at which peak therapeutic effect occurs. As described herein, those skilled in the art will also appreciate that this demonstration of delayed peak effect also indicates that duration of response has been extended.

### Example 3: Effect of MSC Pre-Conditioning on Timing of Peak Therapeutic Effect when Compared to Topical Treatment without MSC Pre-Conditioning

The present Example describes a study that assessed timing of peak therapeutic effect on reduction of Crow's Feet wrinkles for botulinum toxin administered topically (in a nanoemulsion formulation) in combination with microneedle skin preconditioning when compared to a group treated with botulinum toxin administered topically (in a nanoemulsion formulation) without microneedle skin preconditioning.

The study included two test groups, Group A and Group B, of subjects who had moderate to severe Crow's Feet wrinkles. The Crow's Feet area of each subject was treated once topically with a botulinum toxin emulsion (e.g., a nanoemulsion) formulation of comparable dose. Subjects in Groups A (N=26) received no microneedle skin pre-conditioning and Group B (N=9) received skin pre-conditioning with microneedle arrays. Administration of the topical formulation to the skin took about 5 minutes, by which time the topical formulation was fully absorbed into the skin.

The expected effect of a botulinum toxin treatment is to reduce Crow's Feet wrinkles at the site of the botulinum treatment. Wrinkle severity is measured using a five-point wrinkle scale (the Wrinkle Scale): 0=None, I=Minimal. 2=Mild, 3=Moderate, 4=Severe by each of the investigators and subjects. As is commonly accepted in the field, a subject was considered to be a “responder” for purposes of peak effect when the investigator assessed a reduction of two or more points in wrinkle severity on contraction, relative to baseline.

Group A was found to have a peak response rate at one months after treatment; Group B had a peak response rate at three months after treatment.

The study described in this Example documents the unexpected result, described herein, that topical administration of botulinum toxin in a nanoemulsion formulation together with microneedle skin preconditioning, can increase the time at which peak therapeutic effect occurs when compared to topical administration of botulinum toxin in a nanoemulsion formulation without the use of microneedle skin preconditioning.

### Example 4: Impact of Needle Length on Timing of Peak Effect

U.S. Patent Application No. 62/808,274 describes a single dose topical study of the bioavailability of botulinum toxin after topical administration of a botulinum nanoemulsion formulation coupled with microneedle skin conditioning in man. The study tested the impact of varying the length of the microneedles on enhanced botulinum bioavailability in man by measuring wrinkle reduction in the skin following topical treatment with this formulation after skin conditioning with a microneedle array.

The study included three test groups, Group A, Group B and Group C that included subjects who had moderate to severe Crow's Feet wrinkles. The Crow's Feet area of each subject was treated once topically with a topical botulinum formulation that was an emulsion formulation, specifically a nanoemulsion. Subjects in Group A (N=9) received skin pre-conditioning with needle length of 500 μm, subjects in Group B (N=9) skin pre-conditioning with needle length of 800 μm, and subjects in Group C (N=9) skin pre-conditioning with needle length of 1400 μm.

Administration of the topical formulation to the skin in this particular study took about 5 minutes, at which time the topical formulation was fully absorbed into the skin. All subjects were pre-conditioned with the same number of microneedle impressions of a microneedle array prior to application of the botulinum formulation. Doses of botulinum used for Groups A, B, C were matched identically among the subjects.

The expected effect of a botulinum nanoemulsion treatment is to reduce Crow's Feet wrinkles at the site of the botulinum nanoemulsion treatment; such reduction was measured for the different treatments applied in the present Example. Wrinkle severity is measured using a five-point wrinkle scale (the Wrinkle Scale): 0=None, I=Minimal, 2=Mild, 3=Moderate, 4=Severe by each of the investigators and subjects. A responder in this study was a subject who had a reduction in wrinkles severity of two or more points when compared to baseline as assessed by both the investigator and the subject.

This study found that at Baseline, the average severity of the Crow's Feet wrinkles as measured by the Wrinkle Scale was approximately equal across Groups A, B and C. At twelve weeks after treatment, Group A had a responder rate of 36%, Group B had a responder rate of 14%, and Group C had a responder rate of 13%. This study established using shorter microneedles when microneedle skin pre-conditioning unexpectedly increases the bioavailability of a topical, large agent nanoemulsion, and particularly of such a nanoemulsion where the large agent comprises botulinum toxin.

The present Example further establishes that use of such shorter microneedles surprisingly extends peak effect relative to that observed with longer microneedles. Absent the present disclosure, it would have been reasonable to expect that microneedle length would have no effect on timing of peak effect or, if it were to have an effect, that such effect would be to accelerate (rather than delay) peak effect.

The study described above was performed with shorter (e.g., 500 micrometer-long) microneedles vs longer (e.g., 1500 micrometer-long) microneedles. Peak effect was observed substantially later e.g., around 4-8 weeks later) with the shorter microneedles.

### Example 5: Exemplary Emulsion Compositions

In some embodiments, an exemplary macroemulsion may be:

Macroemulsion Formulation

| Component | Weight (g) | Percent (by weight) |
|---|---|---|
| 1349 oil | 22.0 | 22 |
| Tween-80 | 1.0 | 1 |
| Span-65 | 3.0 | 3 |
| Propylparaben | 0.2 | 0.2 |
| Sodium chloride (a) | 0.63 | 0.63 |
| Sodium phosphate dibasic | 0.04 | 0.04 |
| Gelatin | 0.02 | 0.02 |
| Large Agent (e.g., | * | * |
| botulinum toxin and/or | | |
| antibody) | | |
| Isopropyl myristate | 0.62 | 0.62 |
| Purified water (c) | 72.49 | 72.49 |
| Total | 100.22 | 100.00 |

* A person of ordinary skill, in view of the instant specification, could make reasonable adjustments to this and other ingredients depending on the volume, weight, and/or dose of large agent to be utilized.

In some embodiments, a nanoemulsion may be prepared from a premix composition (e.g., by subjecting it to sheer force, such as by microfluidization which, in some embodiments, may be single-pass microfluidization). In some embodiments, an exemplary premix composition may include:

TABLE 2
Exemplary Premix

| % w/w | Ingredient |
|---|---|
| 6.375 | 1349 Oil |
| 9.562 | Polysorbate 80 |
| 0.199 | Propylparaben |
| 63.75 | Isotonic Sodium Chloride Solution |
| 0.199 | Methylparaben |
| 19.92 | Buffer Solution* |
| ** | Large Agent |
| 100 | TOTAL |

*Buffer Solution contains (w/w) 0.199% gelatin, 0.398% sodium phosphate dibasic, 99.4% purified water, pH adjusted to 6.0 ± 0.2 with hydrochloric acid.

An exemplary nanoemulsion formulation, not meant to be limiting, is provided in Table 3.

TABLE 3
Nanoemulsion Formulation

| Component | Weight (g) | Percent (by weight) |
|---|---|---|
| 1349 oil | 3.2 | 3.19 |
| Tween-80 | 4.8 | 4.79 |
| Methylparaben | 0.2 | 0.2 |
| Propylparaben | 0.2 | 0.2 |
| Sodium chloride (a) | 0.63 | 0.63 |
| Sodium phosphate dibasic | 0.04 | 0.04 |
| Gelatin | 0.02 | 0.02 |
| Large Agent (e.g., | * | * |
| botulinum toxin and/or | | |
| antibody) | | |
| Mineral oil | 0.63 | 0.63 |
| Isopropyl myristate | 0.62 | 0.62 |
| White petrolatum | 0.25 | 0.25 |
| Emulsifying wax | 1.87 | 1.87 |
| Purified water (c) | 87.76 | 87.57 |
| Total | 100.22 | 100.00 |

* A person of ordinary skill, in view of the instant specification, could make reasonable adjustments to this and other ingredients depending on the volume, weight, and/or dose of large agent to be utilized.

An exemplary formulation of a botulinum nanoemulsion premix, not meant to be limiting, is provided in Table 4.

TABLE 4
Botulinum Nanoemulsion Recipe (Premix)

| | Amount per | |
|---|---|---|
| % w/w | 400-gram Batch | Ingredient |
| 6.375 | 25.50 | 1349 Oil |
| 9.562 | 38.248 | Polysorbate 80 |
| 0.200 | 0.800 (800 mg) | Propylparaben |
| 63.663 | 254.652 | Isotonic Sodium Chloride Solution |
| 0.20 | 0.800 (800 mg) | Methylparaben |
| 19.21 | 76.84 | GPB Buffer Solution |
| 0.79 | 3.16 | Botulinum toxin diluted in Buffer Solution |
| 100 | 400 | TOTAL THEORETICAL WEIGHT |

* Buffer Solution contains (w/w) 0.199% gelatin, 0.398% sodium phosphate dibasic, 99.4% purified water, pH adjusted to 6.0 ± 0.2 with hydrochloric acid.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating a subject in need of treatment, the method comprising a step of:
administering a plurality of doses of a composition that delivers a large agent having a molecular weight of 100,000 Da or greater to a site on skin of the subject in combination with microneedle skin conditioning (MSC) according to a dosing regimen in which at least two successive doses are separated from one another by a time period of at least one month.

2. The method of claim 1, wherein the time period is at least two months, at least four months, at least six months, at least eight months, at least ten months, or at least twelve months.

3.-7. (canceled)

8. The method of claim 1, wherein the dosing regimen comprises administering at least three doses, separated by at least first and second time periods, wherein the average time period is at least one month.

9. The method of claim 8, wherein the average time period is at least two months, at least four months, at least six months, at least eight months, at least ten months, or at least twelve months.

10.-14. (canceled)

15. The method of claim 8, wherein each of the time periods is the same.

16. The method of claim 15, wherein each of the time periods is at least one month, at least two months, at least four months, at least six months, at least eight months, at least ten months, or at least twelve months.

17.-22. (canceled)

23. The method of claim 1, wherein the administering comprises topically applying the composition to the site.

24. The method of claim 1, wherein the composition comprises a nanoemulsion.

25. The method any one of the preceding claim 1, wherein the composition comprises a macroemulsion.

26. The method of claim 1, further comprising a step of administering a non-irritating penetration enhancing agent.

27. The method of claim 26, wherein the non-irritating penetration enhancing agent is selected from carrier peptides and co-peptides.

28. The method of claim 26, wherein the non-irritating penetration enhancing agent is selected from a cationic peptide and a positively charged carrier with the sequence RKKRRQRRRG-(K)₁₅-GRKKRRQRRR.

29. The method of claim 1, wherein the step of administering comprises performing the MSC (i) prior to administration of the composition to the site, (ii) after administration of the composition to the site, or (iii) concurrently with administration of the composition to the site.

30.-31. (canceled)

32. The method of claim 1, wherein the large agent is a botulinum toxin.

33. The method of claim 32, wherein the step of administering is further in combination with a cosmetic or therapeutic agent.

34. The method of claim 33, wherein the cosmetic or therapeutic agent is selected from the group consisting of anesthetics, collagen, fillers, retinoids, silicone, steroids, and combinations thereof.

35. The method of claim 34, wherein the cosmetic or therapeutic agent is selected from the group consisting of hydrocortisone, retin A, lidocaine, and combinations thereof.

36. The method of claim 1, wherein the large agent is an antibody agent.

37. The method of claim 36, wherein the antibody agent is selected from the group consisting of an anti-TNFα antibody, an anti-CD2 antibody, an anti-CD4 antibody, an anti-IL-12 antibody, an anti-IL-17 antibody, an anti-IL-22 antibody, and an anti-IL-23 antibody.

38. The method of claim 36, wherein the antibody agent is selected from the group consisting of an antibody having epitope binding elements found in one or more of infliximab, adalimumab, golimumab, etanercept, etanercept-szzs, certolizumab pegol, siplizumab, zanolimumab, briakinumab, secukinumab, brodalumab, fezakinumab, ustekinumab and/or guselkumab.

39. The method of claim 36, wherein the step of administering is further in combination with a cosmetic or therapeutic agent.

40. The method of claim 36, further comprising a step of administering a non-irritating penetration enhancing agent.

41. The method of claim 40, wherein the non-irritating penetration enhancing agent is selected from co-peptides and carrier peptides.

42. The method of claim 1, wherein the MSC is accomplished with a device comprising at least one microneedle (MN).

43. The method of claim 42, wherein the device comprises a plurality of MN.

44. The method of claim 42, wherein the device is a patch, a roller, a stamp, or a pen.

45. The method of claim 1, wherein the site is a skin surface (i) overlying a muscle or muscle group, (ii) that contains sweat glands, (iii) that contains sebaceous glands, or (iv) that contains hair follicles.

46.-48. (canceled)

49. The method of claim 42, wherein the MN have a length sufficient to project through the stratum corneum of the skin.

50. The method of claim 42, wherein the MN have a length insufficient to reach nerves in the dermis of the skin.

51. The method of claim 42, wherein the MN are composed of a biocompatible material.

52. The method of claim 42, wherein the MN are composed of a metal, or at least one dissolving polymer.

53. (canceled)

54. The method of claim 1, wherein the MSC comprises administration of 1, 2, 3, 4, 5, 6, 7, 8, 9, 10, 11, 12, 13, 14, 15, 16, 17, 18, 19, or 20 microneedle (MN) impressions, wherein each impression is made in a period of continuous contact between the site and a device that comprises one or more MNs.

55. The method of claim 54, wherein the device is or comprises (i) a stamp that includes the one or more MNs, (ii) a roller that includes the one or more MNs, or (iii) a patch that includes the one or more MNs.

56.-57. (canceled)

58. The method of claim 54, wherein the device comprises a plurality of MNs and MNs of the plurality are arranged in a geometrical pattern.

59. The method of claim 54, wherein the MSC comprises administration of a plurality of the impressions.

60. The method of claim 59, wherein two or more of the impressions are made on approximately the same site.

61. The method of claim 59, wherein two or more of the impressions are made on overlapping sites.

62. The method of claim 54, wherein individual impressions are made on different sites.

63. The method of claim 54, wherein the impressions are made by stamping or by rolling.

64. (canceled)

65. The method of claim 1, wherein the step of administering achieves delivery of the large agent into the skin within about 1 minute, about 2 minutes, about 3 minutes, about 4 minutes, about 5 minutes, about 6 minutes, about 7 minutes, about 8 minutes, about 9 minutes, about 10 minutes, about 5 to about 60 minutes, about 5 to about 12 minutes, about 5 to about 15 minutes, about 15 to about 30 minutes, about 1 hour, about 2 hours, about 3 hours, about 4 hours, about 5 hours, or about 6 hours.

66.-67. (canceled)

68. A method of treating a dermatological disease, disorder, or condition comprising the method of claim 1.

69. The method of claim 68, wherein the subject is suffering from or susceptible to a dermatological disease, disorder, or condition and the administering achieves improvement of one or more features or symptoms of the dermatological disease, disorder, or condition.

70. The method of claim 69, wherein the dermatological disease, disorder, or condition is selected from acne, actinic keratosis, body odor, bromhidrosis, burns, chromhidrosis, dermal infection, eczematous dermatitis, excess sebum-producing disorders, facial wrinkles, hair loss, hyperfunctional facial lines, hyperhidrosis, hyperkinetic facial lines, hyperpigmentation disorders, hypopigmentation disorders, keloids, linear morphea, lupus erythematosus, neck lines, platysma bands, psoriasis, Raynaud's Syndrome, rosacea, scleroderma, skin cancer, unsightly facial expressions, unwanted sweating, and/or combinations thereof.

71. A method of treating or preventing a disease, disorder, or condition selected from acne (e.g., which may be associated with excess sebum production, infection, etc), amyloidosis (e.g., cutaneous amyloidosis), asthma, body odor, burns, cancer (e.g., blood cancer, breast cancer, colon cancer, lung cancer, skin cancer), disorders associated with sun exposure (e.g., actinic keratosis, sunburn, skin cancer such as melanoma, etc), inflammatory conditions (e.g., chronic obstructive pulmonary disorder, Crohn's disease, sweating disorders (e.g., bromhidrosis, chromhidrosis, dermal infection, discoid lupus, drug-induced lupus, eczematous dermatitis, excess sebum-producing disorders, facial wrinkles, hair loss, hyperfunctional facial lines, hyperhidrosis, hyperkinetic facial lines, hyperpigmentation disorders, hyperplasia (e.g., prostate hyperplasia), hypopigmentation disorders, inflammatory bowel disease, keloids, linear morphea, lupus erythematosus, neck lines, neonatal lupus, osteoarthritis, platysma bands, psoriasis, psoriatic arthritis, pulmonary disorders, Raynaud's Syndrome, rheumatoid arthritis, rosacea, scleroderma, skin cancer, systemic amyloidosis, systemic lupus, ulcerative colitis, unsightly facial expressions, unwanted sweating, dyslipidemia, hypercholesterolemia, infection, C. difficile infection, Staphylococcus infection, dystonia, headache, pain, arthritis associated pain, rheumatoid arthritis associated pain, psoriatic arthritis associated pain, osteoarthritis associated pain, certain ophthalmologic conditions, certain urologic conditions, neuromuscular disorders, conditions involving muscular spasm and/or contracture, strabismus, hemifacial spasm, tremor, spasticity such as that resulting from multiple sclerosis, retroorbital muscle, neurologic conditions, migraine or other headaches, Alzheimer's Disease, Parkinson's Disease, or stroke, and/or combinations thereof comprising the method of claim 1.

72. The method of claim 1, wherein the composition is formulated as a lotion, cream, powder, ointment, liniment, gel, or drops.

73. A method of treating a subject in need of treatment, the method comprising a step of:
administering a composition that delivers a large agent having a molecular weight of 100,000 Da or greater to a site on skin of the subject in combination with microneedle skin conditioning (MSC) according to a dosing regimen that achieves a peak effect of the large agent no sooner than about 1 month after the administering.
